(19) United States
(12) Patent Application Publication
(10) Pub. No.: US 2011/0118173 A1
(43) Pub. Date: May 19, 2011

(54) METHOD OF DELIPIDATION OF HDL USING SERUM OPACITY FACTOR TO PREVENT, INHIBIT, AND/OR REVERSE ATHEROSCLEROSIS

(76) Inventors: Henry J. Pownall, Houston, TX (US); Harry S. Courtney, West Memphis, AR (US)

(21) Appl. No.: 12/674,469

(22) PCT Filed: Aug. 22, 2008
(86) PCT No.: PCT/US08/74027

§ 371 Date: Jan. 31, 2011

Related U.S. Application Data
(60) Provisional application No. 60/957,282, filed on Aug. 22, 2007.

(51) Int. Cl. A61K 38/02; A61P 9/10; A61P 3/00; A61P 7/02

(52) U.S. Cl. 514/1.9; 514/7.4

(57) ABSTRACT

This invention relates to delivering a therapeutically active serum opacity factor or an anti-atherosclerotic therapeutic lipoprotein generated from interaction with serum opacity factor to an individual that has or is at risk for atherosclerosis. This can be accomplished by in vivo or ex vivo delivery methods.

### RELATED APPLICATIONS

This application is a national phase filing under 35 USC §371 from PCT International Application Serial No. PCT/US2008/074027, filed Aug. 22, 2008, and also claims priority to U.S. Provisional Patent Application 60/957,282 filed Aug. 22, 2007, all of which applications are incorporated herein in their entirety.

### STATEMENT REGARDING FEDERALLY SPONSORED RESEARCH OR DEVELOPMENT

This invention was made with government support under National Institutes of Health Grant Nos. HL-30914 and HL-56865, and by research funds from the Department of Veterans Affairs. The United States Government has certain rights in the invention.

### TECHNICAL FIELD

The present invention generally concerns at least the fields of physical chemistry, biochemistry, cell biology, molecular biology, and medicine. More specifically, this invention concerns at least the prevention and treatment of atherosclerosis.

### BACKGROUND OF THE INVENTION

Cardiovascular disease (CVD) is a major source of mortality and morbidity and identification of therapies that address its underlying causes is an important public health priority. Foremost among the causes of CVD are dysregulated lipid metabolism that leads to elevated levels of plasma low density lipoprotein (LDL)-cholesterol (C) and to low plasma levels of high density lipoprotein (HDL)-C (Gordon et al., 1997; Miller et al., 1977; Havel et al., 1980). With wider use of the statins, which lower plasma LDL-C, low HDL-C has emerged as the most important lipoprotein disorder for which current therapies are inadequate. HDL is the primary plasma vehicle for reverse cholesterol transport (RCT), the transfer of cholesterol from peripheral tissue including the arterial wall to the liver for recycling or disposal. HDL comprise a core of neutral lipids—cholesteryl esters (CE) and small amounts of triglyceride (TG)—surrounded by a surface monomolecular layer of free cholesterol (FC), phospholipids (PL), and specialized surface binding proteins—apolipoproteins (apos)—mainly apos A-I and A-II (Havel et al., 1980). HDL is an unstable particle residing in a kinetic trap from which it can escape via chaotropic (Mehta et al., 2003), detergent (Pownall, 2005), or thermal perturbation (Mehta et al., 2003; Sparks et al., 1992) Release of lipid-free (LF)-apo A-I is a hallmark of its instability (Mehta et al., 2003; Pownal, 2005; Sparks et al., 1992; Pownall et al., 2007), and is important in two physiological contexts. First, the initiating step in RCT—cellular cholesterol efflux—occurs through the interaction of LF-apo A-I with an ATP-binding cassette (ABC)A1 transporter (Oram et al., 2000). Second, the terminal step in RCT, selective removal of HDL-cholesteryl ester via the hepatic HDL receptor scavenger receptor class B, type I (SR-BI), excludes apo A-I (Acton et al. 1996; Glass et al. 1983), a process that occurs via a delipidation step for which the molecular mechanism is not known.

Serum opacity factor (SOF) is a substance produced by Streptococcus pyogenes that turns mammalian serum opaque, known as opacification (Courtney et al. 1999). SOF is a virulence determinant expressed by approximately half of the clinical isolates of S. pyogenes, a human pathogen that causes a wide spectrum of diseases ranging from pharyngitis to overwhelming invasive infections with high rates of morbidity and mortality (Cunninghan, 2000). The target of opacification is HDL; other lipoproteins are not substantively affected. rSOF opacifies HDL without breaking covalent bonds and is neither a protease nor a lipase (Courtney et al., 2006). The products of SOF activity are buoyant lipid droplets that are devoid of apos and a denser fraction that is rich in apos A-I and A-II. SOF appears to interact with HDL-apos A-I and A-II, thereby triggering the extrusion of HDL lipids, which coalesce into lipid droplets whose growth produces opacification (Courtney et al., 2006).

### BRIEF SUMMARY OF THE INVENTION

An embodiment of the invention is a method of altering reverse cholesterol transport in an individual that has or is at risk for atherosclerosis comprising the step of delivering a therapeutically effective amount of serum opacity factor (SOF) to the individual. In a specific embodiment of the invention, the SOF is recombinant SOF. In another embodiment of the invention, the SOF lacks at least one or more selected from the group consisting of a fibronectin binding site, a leader sequence, Fn-binding repeats and a LPASG anchor. A further embodiment is the method of enhancing reverse cholesterol transport in an individual that has or is at risk for atherosclerosis.

In another specific embodiment of the invention, SOF is delivered by in vivo methods. In a specific embodiment of the invention SOF is injected into the individual. In another embodiment, SOF is injected one or more times into the individual.

In an embodiment of the invention, the delivery method is by ex vivo delivery. In another embodiment of the invention the serum opacity factor is attached to a solid support and the plasma, blood, serum, or isolated HDL of the individual is passed over the support. In another specific embodiment of the invention, the plasma, blood, serum or isolated HDL of the individual is passed over the support in multiple occurrences.

In an additional specific embodiment of the invention, the individual has received, will receive, or is receiving treatment of atherosclerosis. In a specific embodiment the treatment comprises a cholesterol-lowering drug, an anti-platelet drug, an anticoagulant, angioplasty with or without a stent, or surgery.

An embodiment of the invention is a method of generating anti-atherosclerotic therapeutic lipoprotein particles in an individual, comprising the step of delivering an effective amount of serum opacity factor to the individual. In a specific embodiment of the invention the serum opacity factor is recombinant serum opacity factor. In another specific embodiment of the invention the recombinant serum opacity factor is not full-length serum opacity factor. In an additional specific embodiment the recombinant serum opacity factor lacks one or more of the group consisting of a fibronectin binding site, a leader sequence, Fn-binding repeats and a LPASG anchor. In a specific embodiment, the individual has received, will receive, or is receiving treatment for atherosclerosis. In another specific embodiment, the treatment for atherosclerosis comprises a cholesterol-lowering drug, an anti-platelet drug, an anti-coagulant, surgery, angioplasty with or without a stent, or a combination thereof.

Another embodiment of the invention is a method of generating therapeutic lipoprotein particles for an individual with atherosclerosis, comprising the step of delivering an effective amount of the therapeutic lipoprotein particles generated from serum opacity factor, or an effective amount of serum opacity factor to the individual. In a specific embodiment of the invention the serum opacity factor is recombinant serum opacity factor. In another specific embodiment of the invention the recombinant serum opacity factor is not full-length serum opacity factor. In an additional specific embodiment the recombinant serum opacity factor lacks one or more of the group consisting of a fibronectin binding site, a leader sequence, Fn-binding repeats and a LPASG anchor. In a specific embodiment, the individual has received, will receive, or is receiving treatment for atherosclerosis. In another specific embodiment, the treatment for atherosclerosis comprises a cholesterol-lowering drug, an anti-platelet drug, an anti-coagulant, surgery, angioplasty with or without a stent, or a combination thereof.

In another specific embodiment of the invention, SOF is delivered in vivo to generate the anti-atherosclerotic therapeutic lipoprotein particles. In another embodiment, SOF is delivered ex vivo. In an additional embodiment SOF is delivered by attaching it to a solid support, and plasma, blood, serum, or isolated HDL of the individual is passed over the support. In another embodiment the plasma, blood, serum or isolated HDL of the individual is passed over the support in multiple occurrences.

Another embodiment of the invention is a kit for the treatment of atherosclerosis, comprising serum opacity factor housed in a suitable container. In a specific embodiment, the serum opacity factor of the kit is recombinant serum opacity factor. In another specific embodiment, the kit also contains an additional atherosclerosis treatment. In an additional embodiment the additional atherosclerosis treatment comprises a cholesterol-lowering drug, an anti-platelet drug, an anti-coagulant, or a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to characterize the invention, both as to its organization and method of operation, together with further objects and advantages will be better understood from the following description when considered in the context of the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1A-F demonstrates redistribution of 0.5 mg/mL HDL components by recombinant SOF (rSOF) according to size exclusion chromatography (SEC).

FIG. 2A-G demonstrates redistribution of 21 mg/mL HDL components by rSOF according to SEC.

FIG. 3A and FIG. 3B depict the effects of rSOF concentration.

FIG. 4A-E show the effect of HDL concentration on rSOF activity.

FIG. 5A-G demonstrates effects of rSOF on HDL subfractionated according to size. FIG. 5A shows the fractionation of HDL by SEC.

FIG. 6A-D demonstrates effect of rSOF on SEC Profiles of [³H]CE-labeled HDL Subfractions.

FIG. 7A-C shows the kinetics of rSOF-catalyzed HDL opacification.

FIG. 8A-D demonstrates the effect of superphospholipidation on HDL opacification by rSOF.

FIG. 9 demonstrates one embodiment for the mechanism of rSOF-Mediated opacification of HDL.

FIG. 10 shows the effects of rSOF on the SEC profile of HDL subfractions separated according to density.

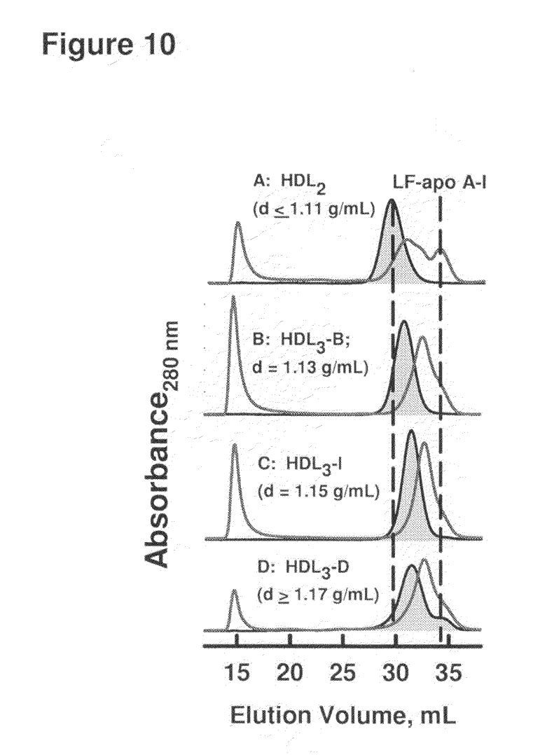

FIG. 11 is a schematic Representation of the Opacification Reaction. rSOF (10 nM) catalyzes the conversion of human HDL (˜20 μM) to a CERM that contains the CE of ˜100,000 HDL particles, a new apo A-II-rich particle called neo HDL, and LF apo A-I. Apo E is the major protein associated with the CERM. The reaction is complete in 90 min. Apos A-I and A-II are shown as black and gray helices respectively.

FIG. 12 (Left) shows images of HDL after mixing with SOF (37° C.). FIG. 12 (Right) demonstrates size distribution as a function of time. Near-linear dimensions of each class-average were measured and weighted by the number of particles contributing to that average. Each point (red) is a class-average. The averages are integrated and interpolated giving a smoothed size distribution; X-axis=particle size; Y-axis=frequency (number of particles in this cluster/total number of particles). The vertical lines in each panel indicated the number-weighted particles sizes at each time point.

FIG. 13 demonstrates opacification kinetics. FIG. 13A is a kinetic analysis of Cryo EM data of FIG. 12 according to a two-parameter exponential fit gave a rate constant, k=(2.42+0.54)×10-2 min-1 (r2>0.96). FIG. 13B is turbidimetric Kinetics of HDL Opacification at various temperatures showing the data (−) and the fitted curve (−). FIG. 13C is an arrhenius plot of opacification; Ea=76.5 kJ/mole. r2>0.99.

FIG. 14 demonstrates effect of rSOF on Ultrastable HDL. FIG. 14A is a SEC profile of HDL before (filled curve) and after treatment (unfilled curve) with 6 M Gdm-Cl. FIG. 14B is a SEC profile of the d<1.21 g/mL (filled curve) and d>1.21 g/mL fractions of HDL after treatment with 6 M Gdm-Cl; the horizontal bar indicates the ultrastable HDL pool. FIG. 14C. is a SEC profile of HDL (0.65 mg/mL) before (filled curve) and after (unfilled curve) treatment with rSOF (1 g/mL for 3 hours). FIG. 14D is a SEC profile of ultrastable HDL (0.65 mg/mL) before (filled curve) and after (unfilled curve) treatment with rSOF (1 g/mL).

FIG. 15 demonstrates kinetics of Opacification of HDL Subfractions. HDL was subfractionated by SEC and the kinetics of opacification determined at 37° C. by measuring the increase in turbidity as a function of time. The HDL particle volumes are indicated adjacent to each curve. The particle volumes were calculated from the elution volumes of each HDL subfraction from a calibrated SEC column. Insert shows the linear relationship between size and the rate of opacification (r2>0.97).

FIG. 16 shows SEC of HDL and Neo HDL. Purified HDL and neo HDL (0.2 mL) were analyzed by SEC using an Amersham-Pharmacia ÄKTA chromatography system with two Superose HR6 columns in tandem and eluted with TBS at a flow rate of 0.45 mL/min; the column effluent was monitored by absorbance (280 nm). The left vertical arrow denotes the void volume. FIG. 17, insert a shows a SDS PAGE of HDL and neo HDL as labeled (5 μg protein/lane); presence and absence of β-mercaptoethanol (βME) is indicated as labeled below the gel. From left to right the samples were protein standards, HDL, neo HDL and apos A-I and A-II as monomer (AII) and dimer (AII₂). FIG. 17, insert b is an agarose (0.79%) gel electrophoresis as labeled, with the direction of migration indicated by right vertical arrow; the horizontal arrow locates the pre β position of neo HDL.

FIG. 17 shows Phospholipid Compositions of HDL, Neo HDL, and CERM with PE, phosphatidylethanolamine; PC, phosphatidylcholine, SM, sphingomyelin.

FIG. 18 shows intrinsic Fluorescence Spectra of HDL and Neo HDL at 22 EC. Excitation was at 280 nm using excitation and emission slits of 4 and 2 nm respectively. The difference between the spectra (ΔIntensity) was calculated after normalizing the peaks to the same intensity.

FIG. 19 is the fluorescence Polarization as a Function of Temperature. HDL, neo HDL, and CERM were labeled with DPH and TMA-DPH and the polarization of fluorescence at their respective spectral maxima was measured.

FIG. 20 shows fluorescence and G. P. of Laurdan as a Function of Temperature. FIG. 21A-C shows fluorescence of Laurdan in HDL, neo HDL, and CERM respectively. Grey arrows show the spectral changes with increasing temperature (16, 22, 27, 32, 37, 42, and 47 EC). FIG. 21D shows temperature dependence of the G. P. of Laurdan in HDL (M), neo HDL (F), and CERM (M).

FIG. 21 shows fluorescence and G. P. of Patman as a Function of Temperature. Labels are the same as in the legend to FIG. 20.

FIG. 22 shows cholesterol efflux from THP-1 macrophages to HDL and neo HDL as labeled.

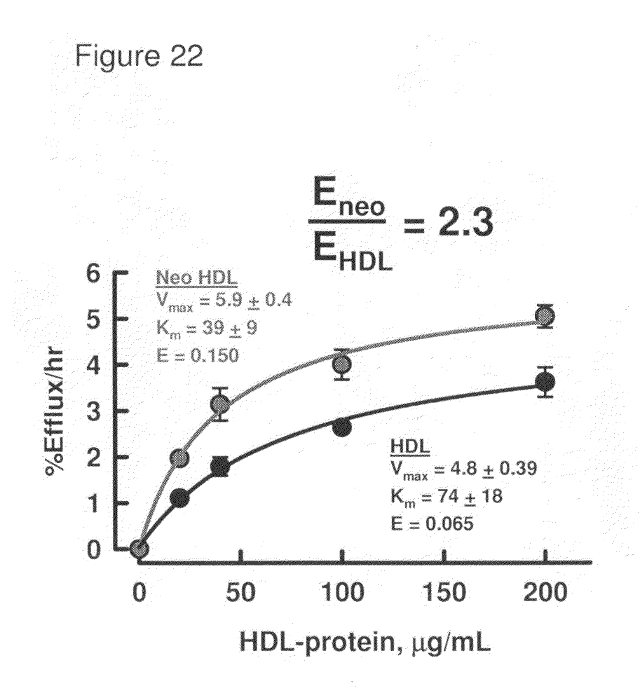

FIG. 23 demonstrates the effects of Dilution on the SEC Profile of apo A-I. Apo A-I (200 μL) in TBS was analyzed by SEC at 5.0, 3.6, 2.3, 1.0, 0.3, and 0.1 mg/mL (left to right). Data are normalized to percent total eluted absorbance at 280 nm.

### DETAILED DESCRIPTION OF THE INVENTION

The present application incorporates by reference herein in its entirety U.S. patent application 60/957,282 filed Aug. 22, 2007.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### I. DEFINITIONS

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “therapeutically effective amount” as used herein refers to an amount that results in an improvement, prevention or remediation of the disease, disorder, or at least one symptom of the disease or condition.

The term “HDL” or “HDL or subspecies thereof” refers to the high density lipoproteins. It is known in the art that high density lipoproteins (HDL) can be fractionated into particulate species defined on molecular size, density, charge, and/or composition. HDL has been resolved into more than twenty-five particle species that differ in charge and molecular size. Each particle is defined by a unique combination of proteins (including apolipoproteins A-I, A-II, A-IV, A-V, D, E, J, L, lecithin:cholesterol acyltransferase, cholesterol ester transfer protein, phospholipid transfer protein, alpha-2 macroglobulin) and lipids (including phospholipid, triglyceride, cholesterol, cholesterol ester, fatty acids), all of which are encompassed here by the use of the term “HDL.”

The term “increase lipid efflux” or “increasing lipid efflux” as used herein refers to an increased level and/or rate of lipid efflux, promoting lipid efflux, enhancing lipid efflux, facilitating lipid efflux, upregulating lipid efflux, improving lipid efflux, enhancing lipid efflux and/or augmenting lipid efflux. In a specific embodiment, the efflux comprises that of phospholipid and cholesterol.

A skilled artisan recognizes that the term “lipid transporter” as used herein refers to a protein or lipoprotein that carries lipids away from peripheral cells into the circulation, and examples include HDL and subspecies thereof, or a mixture thereof. The term “lipid transporter” is also used in the art to refer to, for example, transmembrane proteins that transport cholesterol or phospholipids, from inside a cell to outside the cell. Examples include ABCA1, SR-BI, SR-BII, ABCA4, ABCG5, ABCG8, or a mixture thereof

The term “reverse cholesterol transport” as used herein refers to transport of cholesterol from peripheral tissues to the liver. In a specific embodiment, it refers to efflux of lipid. In specific embodiments, it comprises efflux of cellular cholesterol and/or phospholipid to HDL, and, in further specific embodiments, it comprises HDL delivery of cholesterol ester to the liver, such as for biliary secretion.

The term “anti-atherosclerotic therapeutic lipoprotein particles” or “therapeutic lipoprotein particles” as used herein refers to participles made of lipoproteins that can prevent or treat atherosclerosis. In a specific embodiment, this includes atheropreventive, atheroprotective, and/or atheroregressive particles that produce lesion regression. In a specific embodiment, the particles lower the number or volume of atherosclerotic cells and/or reduce the rate of growth of atherosclerotic cells. In certain specific embodiments, anti-atherosclerotic therapeutic lipoprotein particles comprise HDL, neo HDL, LF Apo A-I and/or CERM.

The term “prevention” or “preventing” as used in relation to a disease herein refers to the use of an effective amount of a compound to prevent the development or the progression of a disease in an individual that is as risk or has the disease. Prevention can minimize, reduce, or suppress the risk of developing a disease state or parameters relating to the disease state, progression or other abnormal or deleterious conditions. In a specific embodiment, the prevention results in delay in onset and/or reduction of intensity of the disease, although in other specific embodiments the prevention results in a complete absence of onset of the disease.

The term “delivering” as used herein is defined as directly or indirectly providing one or more compounds to a destination and includes administering, as for a therapeutic purpose, for example. The delivery may be directly to an individual or indirectly to the individual, such as by ex vivo methods. For example, delivery as used herein also includes methods where SOF or rSOF is not directly administered to the subject, but interacts with the patient through ex vivo methods.

The term “treatment” refers to any process, action, application, therapy, or the like, wherein a mammal, including a human being, is subject to medical aid with the object of improving the mammal's condition, directly or indirectly. In some embodiments treatment is for the prevention of a disease. In another embodiments, one or more symptoms of the mammal's condition is alleviated at least partially.

As used herein, an “individual” is an appropriate subject for the method of the present invention. An individual may be a mammal and in specific embodiments is any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Individuals may also be referred to as “patients” or “subjects”.

### II. GENERAL EMBODIMENTS OF THE INVENTION

Human plasma HDL are important vehicles in reverse cholesterol transport, the cardioprotective mechanism by which peripheral tissue-cholesterol is transported to the liver for disposal. HDL is the target of SOF, a substance produced by Streptococcus pyogenes that turns mammalian serum cloudy. rSOF catalyzes the partial disproportionation of HDL into a CERM and a new HDL-like particle, neo HDL, with the concomitant release of lipid-free (LF)-apo A-I. Opacification is unique; rSOF transfers apo E and nearly all neutral lipids of ˜100,000 HDL particles into a single large CERM whose size increases with HDL-CE content (r ˜100-250 nm) leaving a neo HDL that is enriched in PL (41%) and protein (48%), especially apo A-II. rSOF is potent; within 30 min at 37° C., 10 nM rSOF opacifies 4 ΦM HDL. At respective low and high physiological HDL concentrations, LF-apo A-I is monomeric and tetrameric. CERM formation and apo A-I release have similar kinetics suggesting parallel or rapid sequential steps. According to the reaction products and kinetics, rSOF is a heterodivalent fusogenic protein that uses a docking site to displace apo A-I and bind to exposed CE surfaces on HDL; the resulting rSOF-HDL complex recruits additional HDL with its binding-delipidation site and through multiple fusion steps forms a CERM. rSOF may be a clinically useful and novel modality for enhancing reverse cholesterol transport. With apo E and a high CE content, CERM could transfer large amounts of cholesterol to the liver for disposal via the low density lipoprotein (LDL) receptor; neo HDL is likely a better acceptor of cellular cholesterol than HDL; LF-apo A-I could enhance efflux via the ATP-binding cassette transporter ABCA1.

An embodiment of the invention is the method of altering reverse cholesterol transport in an individual that has, or is at risk for atherosclerosis comprising delivering a therapeutically effective amount of SOF to the individual. In an embodiment of the invention, the method enhances reverse cholesterol transport. In a specific embodiment SOF is not full length and may be missing one or more of the group consisting of a fibronectin binding site, a leader sequence, Fn-binding repeats and a LPASG anchor or any combination thereof. In another specific embodiment, SOF is rSOF. The therapeutically effective amount of SOF may be delivered in vivo or ex vivo. In a specific embodiment, SOF is injected into the individual one or more times. In another specific embodiment, the SOF is attached to a solid support and blood, plasma, serum or isolated HDL of the individual is passed over the support one or more times. In another embodiment of the invention, the individual has received, will receive, or is receiving treatment for atherosclerosis. In another embodiment, the individual is additionally has, will or is currently receiving additional treatment with a cholesterol-lowering drug, an anti-platelet drug, an anticoagulant, angioplasty with or without a stent, or surgery.

Another embodiment of the invention is the method of generating anti-atherosclerotic therapeutic lipoprotein particles in an individual, comprising delivering an effective amount of SOF to the individual. In a specific embodiment of the invention, the SOF is not full length SOF. In another specific embodiment of the invention, SOF is rSOF. In another embodiment of the invention, SOF is missing one or more of the group consisting of a fibronectin binding site, a leader sequence, Fn-binding repeats or a LPASG anchor. In another embodiment the anti-atherosclerotic therapeutic lipoprotein particles are generated in vivo or ex vivo. In another embodiment of the invention, the SOF is injected into the individual one or more times. In another embodiment of the invention, SOF is attached to a solid support and the plasma, blood, serum or isolated HDL of the individual is passed over the support one or more times. In another embodiment of the invention, the individual has received, will receive or is receiving treatment for atherosclerosis that may comprise a cholesterol-lowering drug, an anti-platelet drug, an anti-coagulant, surgery, angioplasty with or without a stent, or a combination thereof.

Another embodiment of the invention is a kit for treatment of atherosclerosis, comprising SOF housed in a suitable container. In a specific embodiment of the invention, SOF is rSOF. In another specific embodiment of the invention, an additional atherosclerosis treatment is also comprised in the kit. In another embodiment of the invention, the additional atherosclerosis treatment comprises a cholesterol-lowering drug, an anti-platelet drug, an anti-coagulant, or a combination thereof. In an embodiment of the invention, the kit additionally comprises an ex vivo support mechanism.

### III. ATHEROSCLEROSIS

The term “atherosclerosis” as used herein includes a combination of changes in the intima of arteries, such changes include, but are not limited to accumulation of lipids, complex carbohydrates, blood and blood products, fibrous tissue and calcium deposits. Atherosclerosis is a disease of the arterial blood vessels. It is characterized by the inflammation of the arterial walls and formation of plaques or lesions within the arteries.

Atherosclerosis develops from the build up of lipids, blood and blood products, complex carbohydrates, fibrous tissue and calcium deposits. This buildup causes plaque and lesions to form within blood vessels. Over time the lesions or plaques can worsen to the point of thrombosis, hematoma, blood clot or hemorrhage. Growth of the plaque or lesions also leads to decreased blood flow and ischemic conditions. In one embodiment, SOF halts the growth of plaques or lesions. In another embodiment, SOF reduces the size of plaques or lesions. In another embodiment, SOF creates anti-atherosclerotic therapeutic lipoprotein particles to halt the growth of or reduce the size of plaques or lesions.

Plasma high density lipoproteins (HDL) are the vehicles for reverse cholesterol transport (RCT), the mechanism by which peripheral tissue-cholesterol is transferred to the liver for recycling or disposal (Cuchel and Rader, 2006). Human HDL comprise free cholesterol (FC), cholesteryl esters (CE), phospholipids (PL), small amounts of triglyceride (TG), and apolipoproteins (apos)—mainly apos A-I, A-II, C, and E (Havel et al., 1980, Gotto and Pownall, 2003). Unlike other plasma lipoproteins, all HDL components are exchangeable by spontaneous (Massey et al., 1984) or protein-mediated mechanisms (Tall, 1995). HDL are further distinguished from other lipoproteins by their instability, which has been identified by chaotropic (Mehta et al., 2003, Pownall et al., 2007), detergent (Pownall, 2005) and thermal perturbations (Mehta et al., 2003, Sparks et al., 1992, Reijngoud and Phillips, 1984), which induces HDL fusion with the concomitant release of lipid-free (LF)-apo A-I. In one embodiment of the invention, SOF enhances RCT. In another embodiment of the invention, SOF creates anti-atherosclerotic therapeutic lipoprotein particles which enhance RCT. In a specific embodiment of the invention, the anti-atherosclerotic therapeutic lipoproteins are HDL, neo-HDL LF Apo A-I and/or CERM. In another embodiment of the invention, RCT enhancement by SOF or SOF created anti-atherosclerotic therapeutic lipoprotein particles treats, prevents, or reduces atherosclerosis.

Atherosclerosis is a progressive disease that usually starts around adolescence, but has been found even in infants (Lawrence, 2007) There are no early warning signs of atherosclerosis; symptoms do not present until atherosclerosis has progressed to a point that it is a serious health risk. Complications of advanced atherosclerosis are coronary artery disease, heart attack, ischemic heart disease, cerebrovascular disease, stroke, transient ischemic attack, peripheral arterial disease, intestinal ischemic syndrome, or aortic aneurysm, for example. In one embodiment of the invention, SOF is used to treat coronary artery disease, ischemic heart disease, heart attack, cerebrovascular disease, stroke, transient ischemic attack, intestinal ischemic syndrome, aortic aneurysm or peripheral arterial disease. In another embodiment of the invention, SOF is used to prevent coronary artery disease, heart attack, ischemic heart disease, cerebrovascular disease, intestinal ischemic syndrome, stroke, transient ischemic attack, or peripheral arterial disease in an individual who is at risk for the relevant disease. One of skill in the art will recognize that the previous listing of complications caused by atherosclerosis is exemplary, and other complications not listed herein can also be treated or prevented by SOF.

Symptoms of atherosclerosis present when the disease is far progressed. Symptoms of atherosclerosis depend on the location of the arteries that the plaques or lesions have developed in. Patients with atherosclerosis near the heart, or coronary atherosclerosis can present with angina, myocardial infarction, chest pressure, chest pain, diaphoresis, nausea, pulmonary adema, hypotension, and vomiting (Lawrence, 2007), for example. Symptoms of atherosclerosis in cerebrovascular arteries can include difficulty speaking, or weakness on one side indicating transient ischemic attack or stroke. Symptoms of atherosclerosis in peripheral limbs can include poor circulation in the arms or legs, pain in the calf muscles when walking, poor wound healing, decreased pulse in the feet, leg numbness or weakness, cold legs or feet, sores on toes, change in color of the limbs, hair loss on the limbs, or changes in nails. Symptoms can be acute or chronic. Symptoms of atherosclerosis in the gut or intestine can include acute symptoms such as mild or severe abdominal pain, forceful bowel movements, abdominal tenderness or distention, blood in stool, nausea, vomiting and fever or chronic symptoms such as abdominal cramps that grow worse over time, unintended weight loss, diarrhea, and bloating. One of skill in the art will recognize the symptoms listed here as exemplary. In one embodiment of the invention, SOF is used to treat patients who present with symptoms of atherosclerosis.

One should not rely on such severe symptoms for a diagnosis of atherosclerosis as these symptoms do not occur until the disease is far progressed. Current diagnoses are routinely done using risk assessments based on family history, blood pressure, cholesterol levels, age, and medical history and can provided an initial indication of atherosclerosis, for example. Many factors are recognized as contributing to the development of atherosclerosis, including genetic factors, hypertension, diabetes, obesity, hypercholesterolemia, stress, inactivity, and smoking (Lawrence, 2007). In one embodiment of the invention, SOF is used to prevent, reduce, or treat atherosclerosis in an individual at risk for atherosclerosis.

Diagnosis of atherosclerosis also depends on the location of the affected artery. Coronary atherosclerosis can be detected by electrocardiogram (ECG), and/or testing for elevated creatine phosphokinase isoenzymes and troponin levels which can result from myocardial cellular damage. Additionally, a stress test can be done by subjecting the patient to physical exercise and monitoring heart rate, myocardial O₂ consumption, blood pressure and ECG.

Magnetic resonance (MR) angiography and computed tomography (CT) angiography can be used to diagnosis atherosclerosis independent of region. More invasive tests for atherosclerosis include cardiac catherterization as a diagnosis of ischemic heart disease. This procedure is invasive and therefore exposes the patient to additional risk. Angiography consists of inserting a catheter into an artery and threading into arterial branches. A injected contrast agent is sometimes used in CT, MR or X-ray to visualize arteries architecture including lumen diameter and intimal-medial thickness.

There are a number of current surgical methods to treat atherosclerosis, and the conditions and diseases caused by advanced atherosclerosis. One of skill in the art will recognize that the methods of surgical treatment vary by location of atherosclerosis, but general exemplary methods include artery bypass and angioplasty with and without a stent. In one embodiment of the invention, SOF is used in combination with surgery to treat atherosclerosis.

Most of the drugs prescribed for atherosclerosis seek to lower cholesterol. Many popular lipid-lowering drugs can reduce LDL-cholesterol by an average of 25-30% when combined with a low-fat, low-cholesterol diet. Lipid-lowering drugs include bile acid resins, “statins” (drugs that inhibit HMG-CoA reductase, an enzyme that controls cholesterol biosynthesis), niacin, and fibric acid derivatives such as gemfibrozil (Lopid). Aspirin helps prevent thrombosis and a variety of other medications can be used to treat the effects of atherosclerosis. Additionally, antiplatelet drugs, histone deacetylase inhibitors, antihyperlipoproteinemic agents, antiarteriosclerotic agents, antithrombotic/fibrinolytic agents, antihypertensive agents, treatment agents for congestive heart failure, antianginal agents or a combination thereof can be used in the treatment of atherosclerosis or a related condition or disease. In one embodiment of the invention, SOF or SOF-generated therapeutic lipoprotein particles are used in combination with one or more other drugs to treat atherosclerosis. In a specific embodiment, SOF or SOF-generated therapeutic lipoprotein particles are used in combination with a cholesterol-lowering drug, an anti-platelet drug, and/or an anti-coagulant

### IV. SERUM OPACITY FACTOR

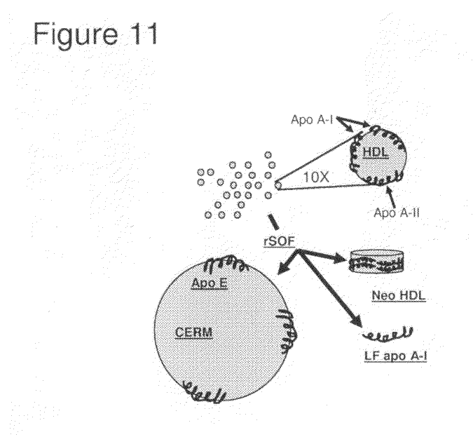

Serum opacity factor (SOF), a protein produced by Streptococcus pyogenes, is a fusogen that causes serum to cloud (Courtney et al., 1999). The opacification reaction is novel if not unprecedented; studies with a recombinant (r) SOF that contains the essential opacification sequence have shown that the plasma reaction is specific to HDL and is associated with disruption of HDL structure and liberation of apos (Courtney et al., 2006). Opacification occurs by a mechanism in which rSOF is a heterodivalent fusogen that catalyzes the disproportionation of HDL into a large CE-rich microemulsion (CERM) and neo HDL, an apo A-II- and PL-rich HDL-like particle, with the concomitant release of LF apo A-I (Gillard et al., 2007). This reaction transfers the CE of >100,000 HDL particles to a single CERM that contains mostly apo E (Gillard et al., 2007). rSOF is potent and catalytic; at 37° C., ˜10 nM rSOF totally opacifies 8 μM HDL in ˜1 hour. rSOF opacifies HDL without breaking covalent bonds; it not an enzyme but rather opacifies via a physical destabilization of HDL (Courtney et al., 2006; Gillard et al., 2007). A schematic for the net reaction is shown in FIG. 11. rSOF (10 nM) catalyzes the conversion of human HDL (˜20 μM) to a CERM that contains the CE of ˜100,000 HDL particles, a new apo A-II-rich particle called neo HDL, and LF apo A-I. Apo E is the major protein associated with the CERM. The reaction is complete in 90 min.

The domains of SOF (SEQ ID NO: 1) are arranged as following; the first 37 amino acids are a leader sequence, following that is the opacification domain which ends at amino acid number 843, Fn-binding repeats follow and end at amino acid 968, and the protein terminates with a LPASG anchor at amino acid 1047. rSOF (SEQ ID NO: 3) contains amino acids 38-843 of SOF (Courtney et al. 2006). The LPASG serves as a cell wall anchor motif. The leading sequence, the Fn-binding repeats, and the LPASG are not needed for opacification.

The rates for rSOF-mediated production of CERM and LF-apo A-I are similar suggesting either concerted (parallel) or rapidly successive steps (Gillard et al., 2007). If concerted, the rate-limiting step might involve simultaneous apo A-I desorption and CE fusion. On the other hand, the reaction could occur in a step-wise mechanism in which one step, formation of CERM or LF-apo A-I is rate-limiting.

The nucleic sequence that codes for SOF, soft, is given in SEQ ID NO: 2, and the nucleic acid sequence for rSOF is given in SEQ ID NO: 4. The NCBI accession number for full length sof is AF082074. One of skill in the art will realize that changes in these sequences may still render an opacification active protein, including, for example, conservative amino acid substitutions and/or truncation of certain regions.

An embodiment of the invention is a method of reducing reverse cholesterol transport in an individual comprising delivering a therapeutically effective amount of serum opacity factor to the individual. In a specific embodiment, SOF is not full length SOF. In a specific embodiment SOF is missing one or more of the leader domain, the LPASG region, and the Fn binding repeat region. In another specific embodiment, SOF is rSOF. In another embodiment, SOF is a synthetic fragment of SOF synthesized by chemical methods or by recombinant DNA methods.

In another embodiment SOF is used to generate anti-atherosclerotic therapeutic lipoprotein particles. In a specific embodiment these lipoprotein particles are atheropreventive, atheroprotective, and/or atheroregressive.

In an embodiment of the invention there is a kit for the treatment of atherosclerosis, comprising serum opacity factor housed in a suitable container. In another embodiment SOF is not full length SOF. In a specific embodiment of the invention, SOF is rSOF. In a specific embodiment of the invention, SOF is a peptide fragment of SOF prepared by recombinant DNA methods or by synthetic chemistry methods. These fragments can be ten or more residues in length with conservative amino acid substitutions that do not substantively reduce the essential opacifying activity; some of these could have higher opacification activity and therapeutic potency. In a specific embodiment of the invention, the kit additionally comprises a solid support for SOF.

In certain cases, derivatives of SOF are employed, including those that are identical to SEQ ID NO: 1, or those that are comprised within SEQ ID NO: 1, some of which may or may not have alterations compared to the corresponding sequence in SEQ ID NO: 1. In specific embodiments, the derivative is at least 1047 amino acids in length, at least 968 amino acids in length, at least 843 amino acids in length, at least 806 amino acids in length, at least 600 amino acids in length, at least 500 amino acids in length, at least 400 amino acids in length, at least 300 amino acids in length, at least 200 amino acids in length, at least at least 170 amino acids in length, at least 165 amino acids in length, at least 160 amino acids in length, at least 155 amino acids in length, at least 150 amino acids in length, at least 145 amino acids in length, at least 140 amino acids in length, at least 135 amino acids in length, at least 130 amino acids in length, at least 125 amino acids in length, at least 120 amino acids in length, at least 115 amino acids in length, at least 110 amino acids in length, at least 105 amino acids in length, at least 100 amino acids in length, at least 90 amino acids in length, at least 80 amino acids in length, at least 70 amino acids in length, at least 60 amino acids in length, at least 50 amino acids in length, at least 40 amino acids in length, at least 30 amino acids in length, at least 20 amino acids in length, or at least 10 amino acids in length. In specific embodiments, the derivative is 70% or more identical to SEQ ID NO: 1, 75% or more identical to SEQ ID NO: 1, 80% or more identical to SEQ ID NO: 1, 85% or more identical to SEQ ID NO: 1, 90% or more identical to SEQ ID NO: 1, 95% or more identical to SEQ ID NO: 1, 97% or more identical to SEQ ID NO: 1, or 99% or more identical to SEQ ID NO: 1. In specific embodiments, the derivative is 70% or more identical to SEQ ID NO: 3, 75% or more identical to SEQ ID NO: 3, 80% or more identical to SEQ ID NO: 3, 85% or more identical to SEQ ID NO: 3, 90% or more identical to SEQ ID NO: 3, 95% or more identical to SEQ ID NO: 3, 97% or more identical to SEQ ID NO: 3, or 99% or more identical to SEQ ID NO: 3. In some embodiments of the invention the SOF derivative or rSOF further comprises a his tag.

### V. PHARMACEUTICAL COMPOSITIONS AND DELIVERY

It is contemplated that the methods and compositions described herein can be used in ex vivo, in vivo, and in vitro applications. For in vivo applications, the therapeutic compositions of the invention can be administered to the patient by a variety of different means. The means of administration will vary depending upon the intended application. As one skilled in the art would recognize, administration of the therapeutic compositions can be carried out in various fashions.

Pharmaceutical compositions of the present invention comprise an effective amount of SOF, SOF-generated therapeutic lipoprotein particles, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains SOF, SOF-generated therapeutic lipoprotein particles, or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards. This includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

SOF or SOF-generated therapeutic lipoprotein particles may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

SOF or SOF-generated therapeutic lipoprotein particles may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include SOF or SOF-generated therapeutic lipoprotein particles, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, SOF may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In an embodiments of the present invention, SOF or SOF-generated therapeutic lipoprotein particles may be formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, SOF or SOF-generated therapeutic lipoprotein particles may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound SOF may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

D. Ex Vivo Delivery Methods

In a specific embodiment of the invention, SOF is covalently attached to a solid support and serum, blood, plasma, or isolated HDL is passed over the immobilized SOF. The SOF is attached to the solid support via reactive species on the solid support that covalently or noncovalently associates with specific amino acids or regions of SOF. SOF could also be bound to a solid support bearing an antibody to SOF epitopes. In another specific embodiment, the serum, blood, plasma, or isolated HDL is passed over the support containing immobilized SOF multiple times. One of skill in the art is well versed on ex vivo delivery methods.

E. Additional Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, an antihypertensive agent, a treatment agent for congestive heart failure, an antianginal agent or a combination thereof.

i. Antihyperlipoproteinemics

In certain embodiments, administration of an agent that lowers the concentration of one of more blood lipids and/or lipoproteins, known herein as an “antihyperlipoproteinemic,” may be combined with administration of SOF or SOF-generated therapeutic lipoprotein particles for cardiovascular therapy, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

1. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

2. Resins/Bile Acid Sequesterants

Non-limiting examples of resins/bile acid sequesterants include cholestyramine (cholybar, questran), colestipol (colestid) and polidexide.

3. HMG CoA Reductase Inhibitors

Non-limiting examples of HMG CoA reductase inhibitors include lovastatin (mevacor), pravastatin (pravochol) or simvastatin (zocor).

4. Nicotinic Acid Derivatives

Non-limiting examples of nicotinic acid derivatives include nicotinate, acepimox, niceritrol, nicoclonate, nicomol and oxiniacic acid.

5. Thyroid Hormones and Analogs

Non-limiting examples of thyroid hormones and analogs thereof include etoroxate, thyropropic acid and thyroxine.

6. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

ii. Antiarteriosclerotics

Non-limiting examples of an antiarteriosclerotic include pyridinol carbamate.

iii. Antithrombotic/Fibrinolytic Agents

In certain embodiments, administration of an agent that aids in the removal or prevention of blood clots may be combined with administration of SOF for cardiovascular therapy, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin).

1. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

2. Antiplatelet Agents

Non-limiting examples of antiplatelet agents include aspirin, a dextran, dipyridamole (persantin), heparin, sulfinpyranone (anturane) and ticlopidine (ticlid).

3. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

iv. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

1. Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

2. Alpha/Beta Blockers

In certain embodiments, an antihypertensive agent is both an alpha and beta adrenergic antagonist. Non-limiting examples of an alpha/beta blocker comprise labetalol (normodyne, trandate).

3. Anti-Angiotension II Agents

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

4. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

5. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

6. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

a. Arylethanolamine Derivatives

Non-limiting examples of arylethanolamine derivatives include amosulalol, bufuralol, dilevalol, labetalol, pronethalol, sotalol and sulfinalol.

b. Benzothiadiazine Derivatives

Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

c. N-carboxyalkyl(peptide/lactam) Derivatives

Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

d. Dihydropyridine Derivatives

Non-limiting examples of dihydropyridine derivatives include amlodipine, felodipine, isradipine, nicardipine, nifedipine, nilvadipine, nisoldipine and nitrendipine.

e. Guanidine Derivatives

Non-limiting examples of guanidine derivatives include bethanidine, debrisoquin, guanabenz, guanacline, guanadrel, guanazodine, guanethidine, guanfacine, guanochlor, guanoxabenz and guanoxan.

f. Hydrazines/Phthalazines

Non-limiting examples of hydrazines/phthalazines include budralazine, cadralazine, dihydralazine, endralazine, hydracarbazine, hydralazine, pheniprazine, pildralazine and todralazine.

g. Imidazole Derivatives

Non-limiting examples of imidazole derivatives include clonidine, lofexidine, phentolamine, tiamenidine and tolonidine.

h. Quanternary Ammonium Compounds

Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

i. Reserpine Derivatives

Non-limiting examples of reserpine derivatives include bietaserpine, deserpidine, rescinnamine, reserpine and syrosingopine.

j. Suflonamide Derivatives

Non-limiting examples of sulfonamide derivatives include ambuside, clopamide, furosemide, indapamide, quinethazone, tripamide and xipamide.

v. Treatment Agents for Congestive Heart Failure

Non-limiting examples of agents for the treatment of congestive heart failure include anti-angiotension II agents, afterload-preload reduction treatment, diuretics and inotropic agents.

1. Afterload-Preload Reduction

In certain embodiments, an animal that can not tolerate an angiotension antagonist may be treated with a combination therapy. Such therapy may combine administration of hydralazine (apresoline) and isosorbide dinitrate (isordil, sorbitrate).

2. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

3. Intropic Agents

Non-limiting examples of a positive intropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

vi. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

vii. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty with or without a stent, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Further treatment of the area of surgery may be accomplished by perfusion, direct injection, systemic injection or local application of the area with at least one additional therapeutic agent (e.g., SOF of the invention, a pharmacological therapeutic agent), as would be known to one of skill in the art or described herein.

F. Combination Therapy

In order to increase the effectiveness of SOF or SOF-generated therapeutic lipoprotein particles, it may be desirable to combine these compositions and methods of the invention with an agent effective in the treatment of vascular or cardiovascular disease or disorder, including atherosclerosis, for example. Exemplary effective agents are discussed supra. In some embodiments, it is contemplated that a conventional therapy or agent, including but not limited to, a pharmacological therapeutic agent, a surgical therapeutic agent (e.g., a surgical procedure) or a combination thereof, may be combined with SOF or SOF-generated therapeutic lipoprotein particle administration. In a non-limiting example, a therapeutic benefit comprises reduced hypertension in a vascular tissue, or reduced restenosis following vascular or cardiovascular intervention, such as occurs during a medical or surgical procedure). Thus, in certain embodiment, a therapeutic method of the present invention may comprise administration of a SOF or SOF-generated therapeutic lipoprotein particle of the present invention in combination with another therapeutic agent.

This process may involve contacting the cell(s) with an agent(s) and SOF or SOF-generated therapeutic lipoprotein particle at the same time or within a period of time wherein separate administration of SOF or SOF-generated therapeutic lipoprotein particle and an agent to a cell, tissue or organism produces a desired therapeutic benefit. The terms “contacted” and “exposed,” when applied to a cell, tissue or organism, are used herein to describe the process by which a therapeutic construct of SOF or SOF-generated therapeutic lipoprotein particles and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both SOF or SOF-generated therapeutic lipoprotein particle and one or more agents, or by contacting the cell with two or more distinct compositions or formulations, wherein one composition includes SOF and the other includes one or more agents.

SOF or SOF-generated therapeutic lipoprotein particle may precede, be co-current with and/or follow the other agent(s) by intervals ranging from minutes to weeks. In embodiments where SOF or SOF-generated therapeutic lipoprotein particle and other agent(s) are applied separately to a cell, tissue or organism, one would generally ensure that a significant period of time did not expire between the time of each delivery, such that SOF or SOF-generated therapeutic lipoprotein particles and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) with SOF or SOF-generated therapeutic lipoprotein particles. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1 week, about 2 weeks, about 3 weeks, about 4 weeks, about 5 weeks, about 6 weeks, about 7 weeks, about 8 weeks, about 1 month, about 2 months, about 3 months, about 4 months, about 5 months, about 6 months, about 7 months, about 8 months, about 9 months, about 10 months, about 11 months, or about 12 months, and any range derivable therein, prior to and/or after administering SOF or SOF-generated therapeutic lipoprotein particles.

Various combination regimens of SOF and one or more agents may be employed. Non-limiting examples of such combinations are shown below, wherein a composition of SOF is “A” and an agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the composition of SOF or SOF-generated therapeutic lipoprotein particles to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

### VI. EXPRESSION SYSTEMS

### A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In a specific embodiment, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra. SOF or rSOF protein may be generated from such a vector and/or expression system given their respective DNA sequences of SEQ ID NO: 2 and SEQ ID NO:4.

i. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference).

Naturally, it will be important to employ a promoter and/or enhancer that effectively directs the expression of the DNA segment in the cell or orgamism chosen for expression. Those of skill in the art of molecular biology generally know the use of promoters and enhancers combinations for protein expression, (see, for example Sambrook et al. 1989, incorporated herein by reference). The promoters employed may be constitutive, inducible, and/or useful under the appropriate conditions to direct high level expression of the introduced DNA segment, such as is advantageous in the large-scale production of recombinant proteins and/or peptides. The promoter may be heterologous or endogenous.

ii. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

iii. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

iv. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

v. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

vi. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. The marker used is not believed to be important, so long as it is capable of being expressed simultaneously with the nucleic acid encoding a gene product. Further examples of selectable and screenable markers are well known to one of skill in the art.

vii. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

### B. Vector Delivery

Suitable methods for nucleic acid delivery for transformation of at least an organism or a cell for use with the current invention are believed to include virtually any method by which a nucleic acid (e.g., DNA) can be introduced into an organism for example, as described herein or as would be known to one of ordinary skill in the art. Such methods include, but are not limited to, direct delivery of DNA such as by electroporation (U.S. Pat. No. 5,384,253, incorporated herein by reference; Tur-Kaspa et al., 1986; Potter et al., 1984, for example. Through the application of such techniques cell(s) or organism(s) may be stably or transiently transformed.

In certain embodiments of the present invention, a nucleic acid is introduced into a cell, or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

### C. Host Cells

As used herein, the terms “cell,” and “cell line,” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli are contemplated as host.

### D. Expression Systems

Numerous expression systems exist that comprise at least a part or all of the compositions discussed above. Prokaryote based systems can be employed for use with the present invention to produce nucleic acid sequences, or their cognate polypeptides, proteins and peptides. Many such systems are commercially and widely available.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

### E. Proteins, Polypeptides, and Peptides

The present invention also provides purified, and in preferred embodiments, substantially purified, proteins, polypeptides, or peptides. The term “purified proteins, polypeptides, or peptides” as used herein, is intended to refer to an proteinaceous composition, isolatable from recombinant host cells, wherein at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

### VII. BIOLOGICAL FUNCTIONAL EQUIVALENTS

As modifications and/or changes may be made in the structure of SOF according to the present invention, while obtaining molecules having similar or improved characteristics, such biologically functional equivalents are also encompassed within the present invention.

A biological equivalent of SOF as used herein is one is a molecule similar to SOF that is able to convert plasma, blood, serum, or isolated HDL into anti-atherosclerotic therapeutic lipoprotein particles. In one embodiment, a SOF biological equivalent still has opacification activity. In another embodiment SOF does not retain opacification activity but retains its anti-atherogenic properties via the non-opacifying particle, neo HDL or via lipid-free apo A-I.

A. Modified Polynucleotides and Polypeptides

Although administration of rSOF is preferable, in some embodiments the SOF composition is a polynucleotide encoding the desired polypeptide or peptide. The biological functional equivalent may comprise a polynucleotide that has been engineered to contain distinct sequences while at the same time retaining the capacity to encode the “wild-type” or standard protein or other polypeptide or peptide of interest. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide may encode a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted. Functional activity, such as the ability to bind lipids, is preferably retained in any natural or synthetic SOF polypeptide or peptide.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

B. Altered Amino Acids

The present invention, in some aspects, may rely on the synthesis of peptides and polypeptides in cyto, via transcription and translation of appropriate polynucleotides. In alternative embodiments, the polypeptide or peptide is synthesized outside a cell, such as chemically. These peptides and polypeptides may include the twenty “natural” amino acids, and, in some embodiments, post-translational modifications thereof. However, in vitro peptide synthesis permits the use of modified and/or unusual amino acids. A table of exemplary, but not limiting, modified and/or unusual amino acids is provided herein below.

TABLE 1
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

C. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents. In a specific embodiment, the key portion comprises lipid binding activity.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al. 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids (Weisshoff et al. 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

### VIII. Kits of the Invention

Any of the SOF or SOF-generated therapeutic lipoprotein particle compositions described herein may be comprised in a kit. The kits will thus comprise, in suitable container means, SOF or SOF-generated therapeutic lipoprotein particle and, in some cases, an additional agent of the present invention. Exemplary additional agents are described elsewhere herein.

The components of the kits may be packaged either in aqueous media or in lyophilized form. SOF or SOF-generated therapeutic lipoprotein particles may also be adhered to a solid support. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing SOF or SOF-generated therapeutic lipoprotein particles, additional agent, and any other reagent containers in close confinement for commercial sale. Such containers may include injection or blow molded plastic containers into which the desired vials are retained.

Compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods for Example 2-9

Materials: 1-palmitoyl-2-oleoyl phosphatidylcholine (POPC) was from Avanti Polar Lipids. HDL was isolated according to its density by sequential flotation of human plasma obtained. The HDL subfractions were separated by SEC in which increasing elution volume (EV) corresponds to decreasing particle size. Fractions from multiple injections (0.5 mL) were pooled and concentrated as needed by placing the sample in a dialysis sack (12,000-14,000 MW exclusion) and placing Sephedex G75 on the outside to remove water. Alternatively HDL (330 mg) was separated into ten fractions by ultracentrifugation in a density gradient between 1.11 to 1.17 g/mL created with KBr. Four fractions HDL₂ and buoyant (B), intermediate (I), and dense (D) HDL₃ with respective densities of less than or equal to 1.11, 1.13, 1.15 and greater than or equal to 1.17 g/mL were selected for testing with SOF. A polyhistidine-tagged, truncated form of soft (SEQ ID NO:1) encoding amino acids 38-843 (SEQ ID NO:3) was cloned and expressed in Escherichia coli (rSOF) and purified by metal affinity chromatography as described previously (Courtney et al. 2006). The effect of SOF on HDL and the generation of large CERM particles has been noted with both native and recombinant poly-histidine tagged SOF indicating that the histidine tag did not alter the biological activity of SOF. Apolipoprotein compositions were determined by SDS PAGE using 15% Tris-Glycine Ready Gels (BioRad). Bands were visualized with Pierce GelCode Blue stain reagent, destained, and recorded by photography. The apo A-I and apo A-II contents of the HDL subfractions were not remarkably different.

Biological labeling of HDL with [³H]CE: [³H]cholesterol (0.1 mCi) was dried under vacuum and redissolved in 100 μL of 95% ethanol. HDL (2 mL, 9.6 mg/mL) was combined with the lecithin:cholesterol acyltransferase (LCAT) activity from the clear zone obtained from the flotation of HDL (4 mL), and the ethanolic solution was added drop-wise while stirring and incubated at 37° C. with mild agitation. Conversion of FC to CE was followed by removing 100 μL at various times, extracting into hexane, and measuring CE formation by TLC. To remove unreacted free cholesterol, the HDL was mixed with LDL (5 mL 5.8 mg/mL) and incubated for 3 hours at 37° C. At the end of the incubation, the density was adjusted to 1.063 g/mL and the LDL floated and removed. The labeled HDL was adjusted to d=1.21 g/mL by the addition of KBr and the [³H]CE-labeled HDL₃ (0.8 μCi/mg protein) isolated by flotation. According to liquid scintillation counting of spots collected after thin layer chromatography, 97% of the radioactivity eluted as CE. The SEC profile of HDL absorbance at 280 nm and [³H]CE radioactivity were nearly the same. [³H]HDL was separated into six fractions by SEC and analyzed by SEC before and after incubation with rSOF.

Analysis of rSOF activity by SEC: various amounts of HDL and rSOF were combined at 37° C. At the end of each incubation, an aliquot (0.2 mL) was analyzed by SEC using an Amersham-Pharmacia ÄKTA chromatography system equipped with two Superose HR6 columns in tandem and eluted with TBS at a flow rate of 0.45 mL/min. The column effluent was monitored by absorbance (280 nm) and the radioactivity of collected fractions. Stokes' radii were calculated from a calibration curve based on protein standards of known Stokes' radius (r). A Stokes volume (V_{S}) was calculated as V_{S}= 4/3πr³.

Composition of rSOF-HDL products: HDL (0.5 and 21 mg/mL respectively) were incubated for 24 hours with rSOF (1 and 4 μg/mL) at 37° C. For preparative chromatography in which the effluent was collected for analysis, a 0.5 mL sample loop was used; pooled fractions from multiple runs were analyzed for protein using a commercial kit (BioRad DC Protein Assay) and for cholesterol, cholesteryl ester, triglyceride, and PC, using commercial kits (Wako Chemicals USA, Inc. Richmond, Va.). Apoprotein composition was determined by SDS PAGE using 4-15% gradient or 18% Tris-Glycine Ready gels (BioRad). Bands were visualized with Pierce GelCode Blue stain reagent, destained, and recorded with the Kodak Electrophoresis Documentation and Analysis System (EDAS 290).

Western blots. SEC fractions were analyzed for apos and SOF by Western blotting. Proteins were resolved on 15% Tris-glycine Ready Gels (BioRad) by SDS-PAGE and transferred to nitrocellulose for immunoblotting. The Western blotting method was essentially that of the Amersham ECL-plus manual (Amersham GE Healthcare). Immunoblots were conducted with HRP-conjugated goat anti-human apo A-II, apo E, apo A-I and apo B from Academy Biomedical (Houston, Tex.). Titration of standard apos gave detection limits of less than 0.1 ng for the three HDL apos A-I, A-II and E. Both the anti-apo E and anti apo A-II detected the apo A-II-E heterodimer. However, relative exposure times indicated that the anti-apo E antibody was about 150 times more sensitive than was the anti-apo A-II antibody. Anti-SOF was a rabbit antiserum to rSOF2ΔFn (Courtney et al. 2006) and was detected with an HRP-conjugated goat anti-rabbit IgG second antibody (BioRad).

### Example 2

### Compositions of the Products of RSOF and HDL

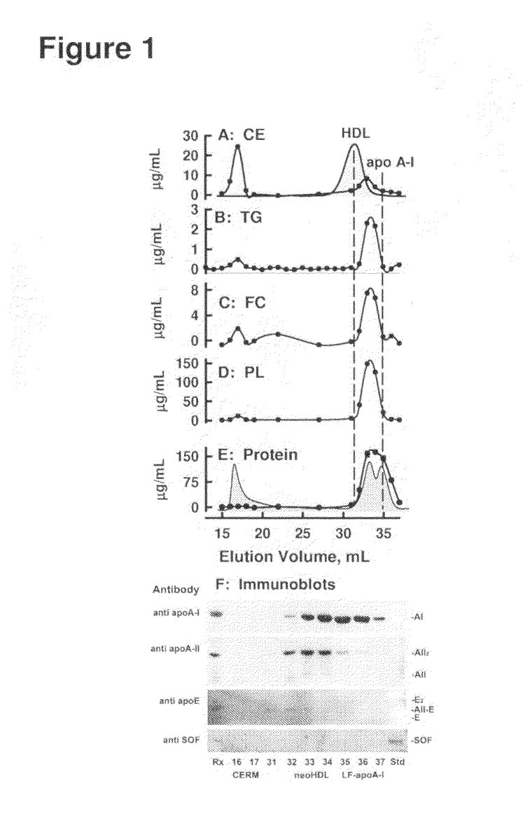

HDL (0.5 mg/mL) elutes from a SEC column as a single peak with an elution volume corresponding to a molecular volume of ˜670 nm³. After incubation of HDL (0.5 mg/mL) and rSOF (1 μg/mL) for 24 hours at 37° C. the initially clear HDL solution was translucent. The absorbance profile for HDL is shown as the filled curve in FIG. 1A. The products were separated by SEC and the collected fractions analyzed for cholesteryl ester (CE), triglyceride (TG), free cholesterol (FC), and phospholipid (PL) (FIG. 1A-D respectively; Table 2); FIG. 1E is the absorbance (280 nm) of the column effluent (filled curve) and total protein according to direct analysis of collected fractions (circle). The standard is 10 ng rSOF and Kaleidoscope molecular weight standards (BioRad). The fraction appearing in the void volume contained ˜60% of the total CE that eluted from the column, 4% of the PL, and only ˜1% of the protein. This fraction is rich in neutral lipids (NL ˜73%), particularly CE (Table 2); accordingly, the particles in this fraction are referred to as cholesteryl ester-rich microemulsion (CERM). Another new HDL-like particle eluting slightly later than HDL contained 87% of the PL applied to the column, ˜60% of the protein, ˜75% of the TG, and ˜80% of the free cholesterol. Protein and PL accounted for nearly 90% of its composition; according to its composition and size (Table 2), the particles in this fraction are called neo HDL; relative to HDL, neo HDL is PL and apo A-II-rich (FIG. 1D; FIG. 1F; Table 2) and one third smaller (˜450 nm³). Calculation of the stoichiometry of CE, TG, and PL in HDL and neo HDL shows that the number of CE molecules per particle is reduced by rSOF but that PL content is conserved (Table 2). Immunoblot analysis revealed the presence of apo E in the leading edge of the neo HDL (FIG. 1F). The last peak to elute from the column was lipid-free and had an elution volume identical to that of an authentic sample of apo A-I, which has a measure Stoke's volume of ˜230 nm³ (FIG. 1E); immunoblot analysis showed that this peak contained apo A-I but no apo A-II (FIG. 1F). Thus, this fraction is lipid-free (LF)-apo A-I.

TABLE 2
Compositions of Products Formed from rSOF at High and Low HDL Concentrations^{a}

| | analytes (% composition) | | | | | (NL/particle)/(PL/particle) | | |
|---|---|---|---|---|---|---|---|---|
| fraction | PL | EC | CE | TG | protein | CE/PL | TG/PL | NL/PL(M/M) |

low concentration of HDL (0.5 mg/mL)

| | | | | | | | | |
|---|---|---|---|---|---|---|---|---|
| HDL | 26.2 | 2.5 | 19.8 | 4.2 | 47.3 | 38/44 = 0.86 | 6/44 = 0.14 | 1.00 |
| CERM | 19.5 | 3.1 | 65.8 | 7.9 | 3.7 | 3.9 | 0.34 | 4.24 |
| neo HDL | 40.8 | 2.1 | 2.9 | 6.6 | 48.0 | 4/44 = 0.09 | 6/44 = 0.14 | 0.23 |

high concentration HDL (21 mg/mL)

| | | | | | | | | |
|---|---|---|---|---|---|---|---|---|
| HDL | 24.4 | 3.0 | 17.3 | 4.4 | 51.0 | 33/41 = 0.80 | 6/41 = 0.15 | 1.05 |
| CERM | 12.4 | 5.2 | 58.0 | 21.9 | 2.5 | 5.4 | 1.5 | 6.9 |
| (apo A-I)ₐ | 4.3 | 0.6 | 1.8 | 1.8 | 91.4 | n.d. | n.d. | n.d. |
| neo HDL | 32.6 | 1.8 | 2.2 | 4.8 | 58.5 | 3/35 = 0.0 | 4/35 = 0.11 | 0.20 |

^{a}Assumes that CE = 650 Da, TG = 885 Da, PL = 750 Da, HDL = 125 kDa, and neo HDL = 80 kDa.
NL = CE + TG;
n.d., not determined

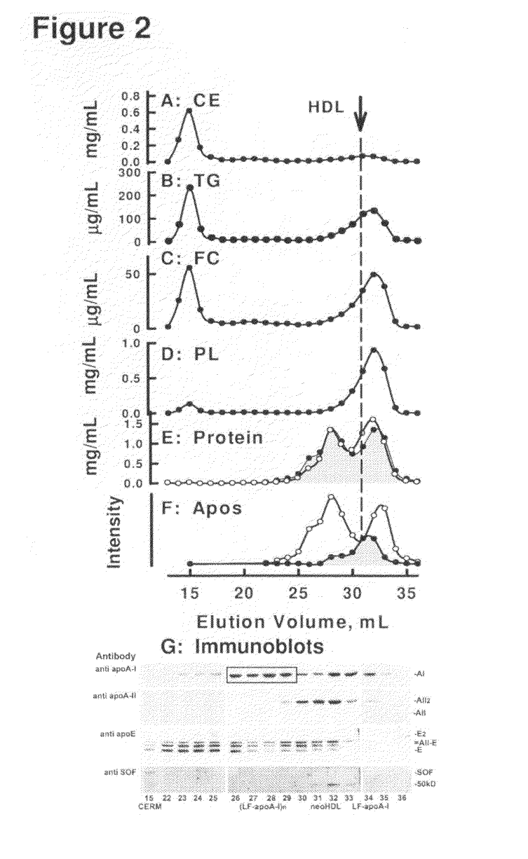

Similar studies at higher concentrations of HDL and rSOF permitted the visualization of low abundance components and revealed a change in the state of association of LF-apo A-I. Incubation of rSOF (4 μg/mL) with whole human HDL (21 mg/mL; 24 hours, 37° C.) rendered the clear yellow HDL solution totally opaque. The products were separated by SEC and the collected fractions analyzed for cholesteryl ester (CE), triglyceride (TG), free cholesterol (FC), and phospholipid (PL) (FIG. 2A-D respectively). FIG. 2E shows the total protein according to direct analysis (open circle) and relative protein content according to quantitative densitometric immunoblotting of SDS PAGE (circle, gray fill). FIG. 2F is Apo A-I (open circle) and apo A-II (circle, gray fill) according to immunoblot densitometry and FIG. 2G is the immunoblot analysis of collected fractions as labeled. The relative amounts loaded per lane were 20 μL (fraction 15), 10 μL (fractions 22-25 and 34-36), and 34, (fractions 26-33). Both CERM and neo HDL appeared in the SEC and had compositions similar to those observed at low HDL concentrations (FIG. 2; Table 2). The CERM contained ˜80% of the total CE in the reaction products. The corresponding values for the other components decreased in the order TG (43%)>FC (38%)>PL (8%)>protein (0.5%). The protein profiles obtained by chemical analysis and quantitative immunoblot analysis of apos A-I and A-II were nearly identical (FIG. 2E) so that the SEC apo distribution can be assigned with confidence. These showed coelution of all apo A-II but not apo A-I with the neo HDL (FIG. 2F). Immunoblotting also revealed the occurrence of apo E in the CERM and in the larger neo HDL particles (FIG. 2G). Apo E was also prominent on larger particles that eluted later than the void volume (22-25 mL). In one embodiment, these are intermediates destined to become CERM. At these higher reactant concentrations, rSOF was observed in the CERM and as a possible ˜50 kDa fragment in neo HDL; this was consistently observed, and in one embodiment is due to proteolysis during the long (24 hour) incubation. Finally, in contrast to the monomeric LF-apo A-I found at low reactant concentrations, at high concentrations, a shoulder and peak eluting at ˜26 and ˜28 mL were practically lipid-free. These particles were ˜1800 and ˜790 nm³ respectively; lipid and immunoblot analysis showed the protein to be mostly lipid-free apo A-I (FIG. 2G boxed) with a trace of apo E that are likely the overlapping edges of peaks for neo HDL and earlier eluting species. Based on their respective elution volumes, these particles are apo A-I octamers and tetramers.

### Example 3

### Effect of RSOF Concentration

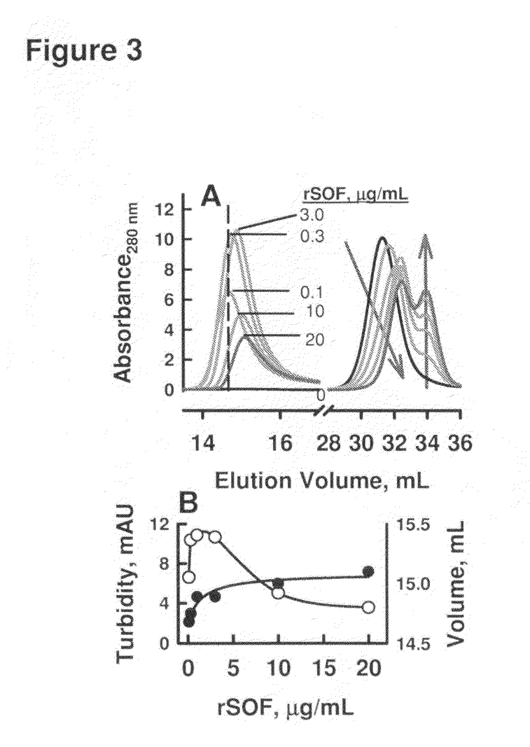

HDL (0.25 mg/mL) was incubated with various concentrations of rSOF for 0.5 hours at 37° C., quenched by immersion in ice for ˜10 min, and analyzed by SEC (FIG. 3A). HDL profile in the absence of rSOF is shown by the black line. rSOF concentrations are indicated with the profile at the highest concentration (20 μg/mL) shown by a dark gray line. Dashed vertical line denotes the void volume. Arrows indicate the direction of the shift in the SEC profiles with increasing rSOF. FIG. 3B depicts cholesteryl ester-rich microemulsion (CERM) turbidity (open circle; splined, smoothed curve) and peak elution volume (closed circle; hyperbolic 3-parameter fit) as a function of rSOF concentration. As the rSOF concentration was increased from 0.1 to 20 μg/mL, the starting HDL SEC profile gradually shifted to the smaller neo HDL with a simultaneous increase in the magnitude of the peak for LF-apo A-I (FIG. 3A). Over the same rSOF concentration range, the peak elution volume for the CERM shifted from the void volume (14.7 mL) at 0.1 μg/mL rSOF into the included volume as the rSOF concentration was increased (FIG. 3A; FIG. 3B); at 20 g/mL rSOF, the absorbance peak for the CERM appeared at 15.1 mL. The SEC are reproducible; the elution volume of the CERM from multiple injections (n=14) was 15.00±0.0157 (SE). Thus, the sizes of the particles that comprise the CERM decrease with increasing rSOF concentration. Consistent with the production of smaller particles, the absorbance due to opacification decreased as the rSOF concentration was raised from 0.3 to 20 μg/mL (FIG. 3B).

### Example 4

### Effect of HDL Concentration on the Opacification Reaction

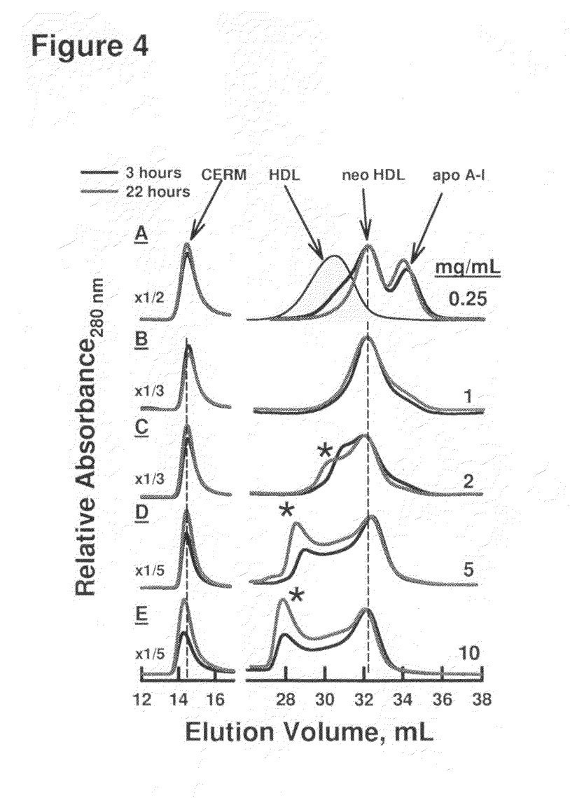

The effect of HDL concentration on opacification by rSOF was also assessed (FIG. 4). Various concentrations of HDL in 1 mL were combined with 4 μg rSOF and incubated at 37° C. for 3 (black line) and 22 hours (grey line) and analyzed by SEC. Panels in FIG. 4A-E are the SEC of HDL at 0.25, 1, 2, 5, and 10 mg/mL HDL-protein. The gray-filled curve in FIG. 4A is the SEC profile for HDL without incubation with rSOF. The absorbance due to the light scattering in the void volume (peak elution ˜14 mL) is much greater than that of the protein absorbance in the right-hand panel; thus, the absorbances in the left-hand panels have been multiplied by the fractions as shown. Asterisks denote the peaks for apo A-I oligomers. At low concentrations, HDL eluted as a single broad peak (FIG. 4A) that was replaced by CERM, neo HDL and LF-apo A-I after incubation with rSOF at 37° C. for 3 or 22 hours. Between 1 and 10 mg/mL HDL the magnitude of the peak for the CERM grew while a new peak appeared, first as a shoulder at 2 mg/mL HDL and then at higher concentrations as a prominent peak at ˜28 mL (marked by asterisks in FIG. 4B-E); the magnitudes of this peak, identified as LF-apo A-I oligomers (FIG. 2) and the peak in the void volume were higher after 22 hours. Thus, rSOF converts HDL to CERM and apo A-I oligomers in a concentration- and time-dependent way.

### Example 5

### Speciation of RSOF Activity

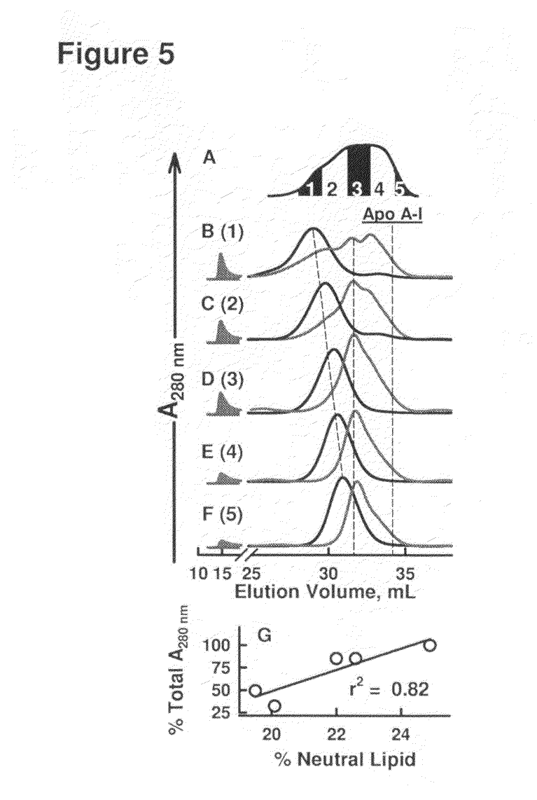

Analysis of the effects of rSOF on HDL subfractions separated according to size showed major differences in the SEC profiles, particularly the amount of material in the void volume (FIG. 5). FIGS. 5B-F are the SEC analysis of HDL subfractions (1.2 mg/mL) from FIG. 5A before (black line) and after (grey line) 24 hour incubation with rSOF (1.1 μg/mL) at 37° C. Relative to fraction 1 (=100%), the peak heights for the void volumes were 85, 85, 49, and 32% for fractions 2 to 5 respectively. FIG. 5G depicts the void volume peak area (gray filled) as a function of neutral lipid content (CE+TG; Table 2) of the starting HDL subfractions. As expected, chemical analysis of the fractions showed that the NL content of the HDL subfractions increased with increasing particle size (Table 3). The amount of material eluting in the void volume, based on integrated absorbance, increased with increasing particle size (FIG. 5B-F) and was highly correlated with the sum of the NL (CE+TG) content of the starting HDL particles (FIG. 5G; r²=0.82). Incubation of rSOF with all HDL subfractions gave rise to a neo HDL with essentially the same particle size. Similar effects were observed with HDL isolated according to density with the SEC profiles of the larger HDL₂ after incubation with rSOF being similar to that of the largest HDL fraction isolated by SEC and the corresponding HDL₃ profile being similar to those of the smaller SEC HDL fractions.

TABLE 3
Composition of HDL and Its Subfractions Separated by Size^{α}

| | % PL | % FC | % CE | % TG | % Protein |
|---|---|---|---|---|---|
| Total | 24.4 ± 1.2 | 2.95 ± 0.26 | 17.3 ± 1.0 | 4.42 ± 0.31 | 51.0 ± 5.5 |
| HDL | | | | | |
| 1 | 29.8 ± 1.0 | 2.63 ± 0.08 | 21.2 ± 1.1 | 3.70 ± 0.26 | 42.7 ± 2.7 |
| 2 | 26.8 ± 4.2 | 2.39 ± 0.11 | 19.3 ± 0.9 | 3.28 ± 0.14 | 48.2 ± 2.2 |
| 3 | 28.9 ± 0.7 | 2.12 ± 0.21 | 19.0 ± 0.3 | 3.04 ± 0.08 | 46.9 ± 0.6 |
| 4 | 27.0 ± 1.2 | 1.92 ± 0.15 | 16.8 ± 0.5 | 2.65 ± 0.10 | 51.7 ± 2.7 |
| 5 | 25.8 ± 1.1 | 1.81 ± 0.09 | 17.4 ± 1.3 | 2.73 ± 0.09 | 52.3 ± 2.1 |

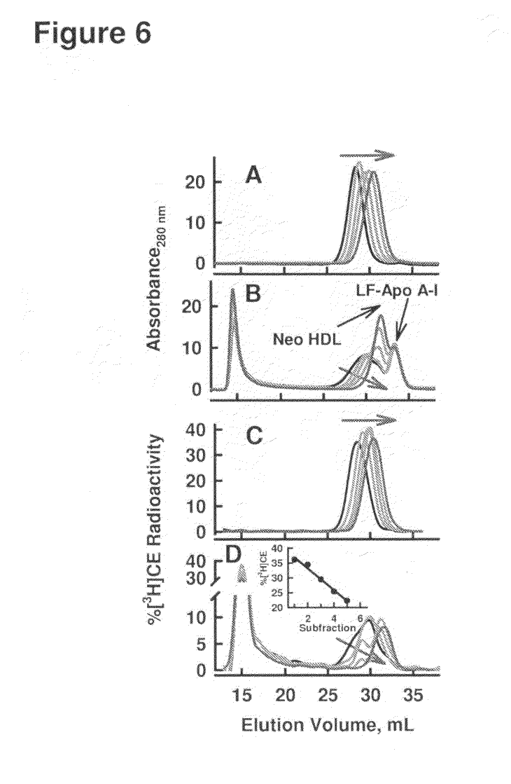

^{α}The HDL subfractions correspond to fractions 1-5 of FIG. 5.

[³H]CE-labeled HDL was used to follow the redistribution of HDL-CE into CERM and neo HDL. [³H]CE-labeled HDL separated into six fractions by SEC, which were analyzed by SEC before and after incubation with rSOF. HDL (0.5 mg/mL) and rSOF (1 μg/mL) were incubated at 37° C. for 3 hours and analyzed by SEC. Elution profiles are shown for absorbance at 280 nm (FIG. 6A, FIG. 6B) and radioactivity (FIG. 6C, FIG. D). FIGS. 6A and C depict SEC profiles of HDL subfractions isolated by SEC. FIGS. 6B and D depict SEC analysis of HDL after incubation with rSOF. In each panel, the largest and smallest HDL subfraction appear as black and gray curves respectively. Gray arrows point in the direction of decreasing size. Peaks for neo HDL and LF-apo A-I are as indicated by black arrows (FIG. D, insert). %[³H]CE in neo HDL with decreasing size of the starting HDL (5 is the smallest). Line of regression includes all data (r2>0.98). According to the SEC absorbance profiles, rSOF converted all subfractions into CERM, LF-apo A-I, and neo HDL, and with decreasing HDL size, the fraction of HDL-protein converted to neo HDL increased while the amount of LF-apo A-I formed was constant (FIG. 6B). The isosbestic point at ˜31 mL is suggestive of a simple two-state system HDL and neo HDL, with the amount of LF-apo A-I, the a third component formed, being constant across all fractions. The absorbance and HDL-[³H]CE profiles of each starting subfraction were nearly coincident (FIG. 6A; FIG. 6C). The product profile as assessed by HDL-[³H]CE radioactivity was similar to that for absorbance except for the absence of the peak for LF-apo A-I (FIG. 6B, FIG. 6D). Despite the profound shift in the HDL-[³H]CE profile of neo HDL with decreasing HDL size (FIG. 6D, compare black and gray curves), the fraction of [³H]CE in neo HDL declined with decreasing size. Also, the fraction of [³H]CE in neo HDL decreases with decreasing size of the HDL from which it is derived (FIG. 6D, insert). These data corroborate the compositional data (FIGS. 1 and 2) on the distribution of HDL-CE and show that the labeling method described above gives a product for which the distribution of [³H]CE is similar to that of chemically determined CE. Moreover, these data show that rSOF releases the same amount of LF-apo A-I from all HDL subfractions but that the smaller more protein-rich subfractions that contain less PL form neo HDL that have less CE.

### Example 6

### Kinetics of HDL Opacification

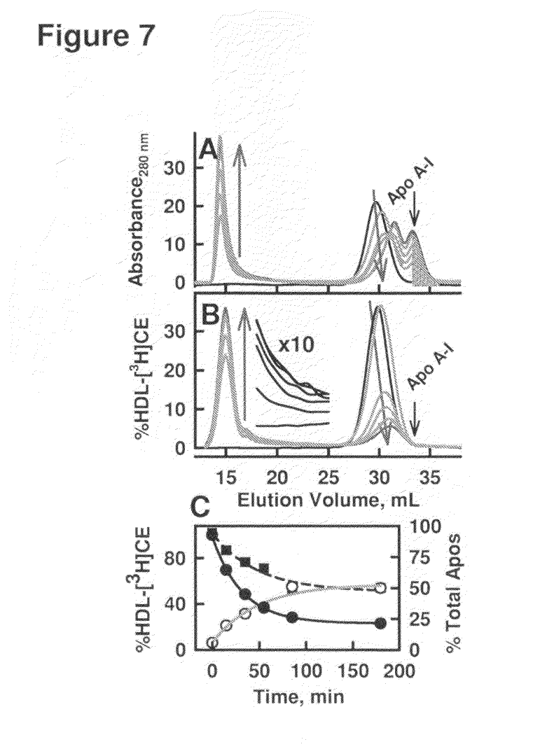

rSOF was incubated with HDL-[³H]CE and the redistribution of absorbance and [³H]CE was followed with time by SEC. HDL-[³H]CE (0.5 mg/mL) was incubated with rSOF (1 μg/mL) at 37° C. for various times as labeled, cooled with wet ice and analyzed by SEC in which the effluent was monitored by absorbance at 280 nm (FIG. 7A) and by the radioactivity (FIG. 7B) of the collected fractions; curves for 0 and 180 min are black and gray respectively. The gray arrows indicate the shift in the adjacent profiles with time=0, 15, 35, 55, 85, 180 min. FIG. 7A shows absorbance, FIG. 7B shows radioactivity, and the insert is the radioactivity multiplied by 10. FIG. 7C depicts the kinetics of appearance of LF-apo A-I calculated as twice the percent of total protein absorbance in the double shaded portion in A (open circle); kinetics of disappearance of HDL calculated from the total protein absorbance minus absorbance due to LF-apo A-I (filled square); kinetics of disappearance of HDL-associated [³H]CE (filled circle). Based on the disappearance of HDL-[³H]CE and protein absorbance respectively, k_{CERM}=(3.2±0.008)×10⁻² (r²>0.99) and k_{AI}=(2.1±0.006)×10⁻² min⁻¹ (r²>0.96). Before incubation, SEC analysis showed co-elution of protein absorbance and radiolabel (FIG. 7A, FIG. 7B, black curves). Following the addition of rSOF to HDL, the magnitude of the peak absorbances for CERM, neo HDL, and LF-apo A-I rose while that for HDL fell. Although not apparent in the absorbance profile, with a ten-fold amplification, the radiolabel analysis reveals the early appearance of CE in fractions between the CERM and the neo HDL that level off at t>85 min (FIG. 7B insert). Based on the rate of increase in the peak for LF-apo A-I, a first order rate constant was calculated as kₘ=(2.1±0.006)×10⁻² min⁻¹ (r²>0.96). The rate constant for the formation of CERM, calculated from the rate of transfer of HDL-associated [³H]CE to CERM was k_{CERM}=(3.2±0.008)×10⁻² (r²>0.99). Additional rSOF (1 μg/mL) and incubation for another 7 hours did not change the final elution profile. Given that k_{AI}˜k_{CERM}, CERM formation and release of LF-apo A-I are either concerted processes or occur in rapid succession. As expected, the reaction rates increase with temperature and yield linear Arrhenius plots between 25 and 42° C.

### Example 7

### RSOF does not Displace Apo A-I from Superphospholipidated (SPLD) HDL

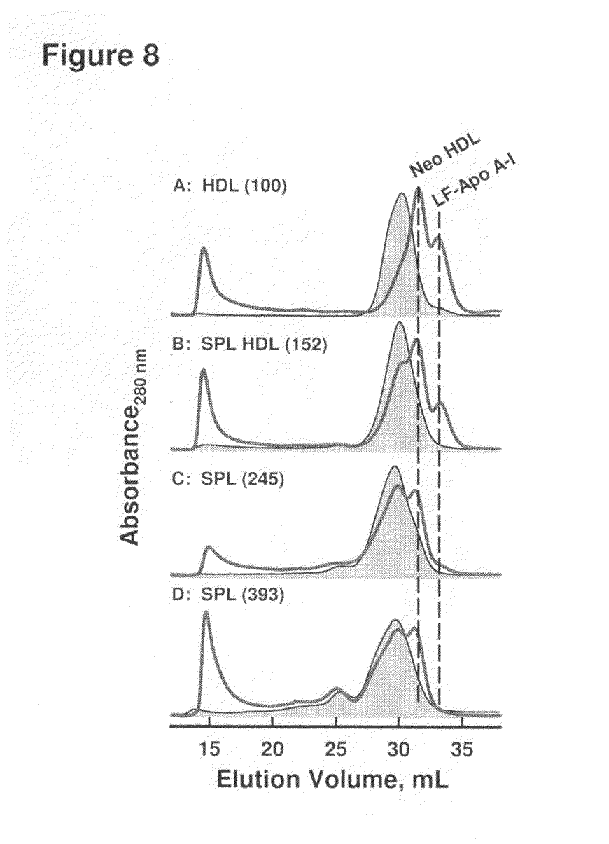

Given that PL are the essential apo-associating components of HDL, HDL-PL was tested to see if increased HDL-PL would stabilize HDL against rSOF. Using a modified detergent dilution method (Pownall, 2007), the PL content of HDL was increased by the addition of POPC. Sodium cholate and 1-palmitoyl-2-oleoyl phosphatidylcholine (POPC) were combined in TBS to give final concentrations of 52.6 and 26.3 mM respectively. Different amounts of cholate/POPC solution were added to HDL (1.3 mg/mL) with rapid stiffing. The final concentration in the mixture did not exceed the critical micelle concentration of sodium cholate (˜15 mM). The superphospholipidated (SPLd) HDL (1 mg/mL) were exhaustively dialyzed against TBS and reacted with rSOF (1 μg/mL). The SEC profiles are before (FIG. 8, grey filled curve) and after (FIG. 8, gray line) incubation with rSOF. FIG. 8A is the control HDL, FIGS. 8B-D are the SPLd HDL containing increasing amounts of PL as labeled. Vertical lines in FIG. 8 locate neo HDL and LF-apo A-I.

Relative to control HDL-PL=100, the PL contents of the SPLd species were 100±0.2, 152±0.05, 245±0.06, and 393±0.01. According to SEC analysis, SPLd HDL was slightly larger than HDL; this was confirmed by non denaturing gradient gel electrophoresis, which showed a shift in HDL particle mass from ˜150 to ˜350 kDa. As expected, incubation of HDL (0.8 mg/mL) with rSOF (1 μg/mL) produced CERM, neo HDL, and LF-apo A-I (FIG. 8). Similar incubations with SPLd containing 1.5-fold HDL-PL reduced the amount of neo HDL formed and LF-Apo A-I released (FIG. 8B), and SPLd HDL containing more than 1.5 fold HDL-PL formed even less neo HDL and released no LF-apo A-I. Thus, the addition of PL partially stabilizes HDL against CERM formation and especially the release of LF-apo A-I (FIG. 8A-C). The increase in the void volume peak (FIG. 8D) in the presence of 4-fold HDL-PL is likely due to a small amount of CERM superimposed on PL multilayers (Pownall 2006).

### Example 8

### Apo A-I Self-Associates at Physiological Concentrations

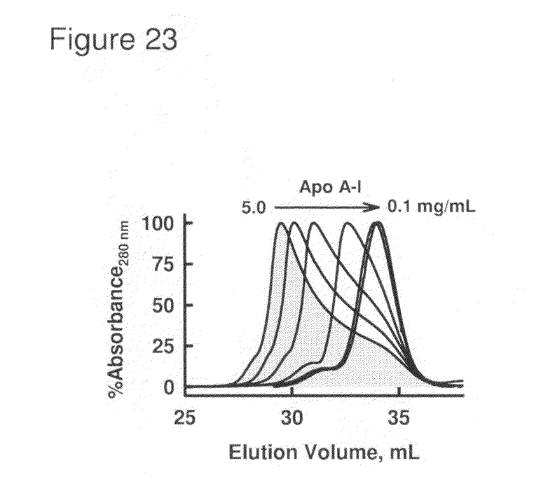

Apo A-I at various concentrations was analyzed by SEC. At 0.1 and 0.3 mg/mL apo A-I elutes as a single peak (EV ˜34 mL; Stokes volume=230 nm³). As the concentration increased to 5 mg/mL, the EV shifted to ˜29.5 mL (Stokes volume=864 nm³), which corresponds to tetrameric apo A-I. In each case, the injected 0.2 mL was diluted to at least 2 mL by the chromatography so that the concentration at which apo A-I self-association occurs is 10% of that injected, i.e., >0.03 mg/mL. Thus, apo A-I forms higher order oligomers in a concentration-dependent way, and is monomeric only at injected apo A-I concentrations less than or equal to 0.3 mg/mL which corresponds to an eluted concentration less than or equal to 0.03 mg/mL (FIG. 23).

### Example 9

### Significance of Examples 2-8

Opacification Reaction: rSOF selectively delipidates HDL via a limited HDL disproportionation, i.e., partial segregation of HDL lipids and proteins to form three major products CERM, neo HDL, and LF-apo A-I. At reaction completion, neo HDL contain most of the PL and protein, with the remainder of the protein occurring as LF-apo A-I. Based on changes in composition, each neo HDL is derived from a single HDL, which is 19.8% CE and 47.3% protein (Table 2). According to the tabular data that was used to create FIG. 1A and FIG. 1E, rSOF transfers 90% of the HDL-CE to the CERM and converts 40% of the protein to LF-apo A-I thereby reducing the particle volume by (0.9×19.8%˜18)+(0.4×47.3%˜19%)=37% leaving a particle that has 63% of the mass of the starting HDL. This value compares favorably with the ratios of the volumes of neo HDL and HDL measured by SEC (100%×450 nm³/650 nm³=69%) and is thus consistent with each neo HDL being derived from a single HDL. This conclusion is supported by stoichiometric considerations. According to its mass (125 kDa) and composition, each HDL contains 38, 6, and 44 CE, TG, and PL molecules compared to 80 kDa, 4, 6, and 44 respectively for neo HDL (Table 2). Thus, the number of PL molecules per particle is conserved during opacification (Table 2). NL give lipoproteins their spheroidal shape through phase separation into a central core. Neo HDL contains only 4 and 6 CE and TG per particle respectively. Phase separation occurs when the CE and TG content exceeds 3 mol % of PL (Hamilton et al., 1983). For neo HDL, this corresponds to 1 molecule of each leaving 3 CE and 5 TG molecules respectively per neo HDL particle. Theoretically, this would be high enough to produce segregation into a lipid lens but would not give rise to a prominent core so that neo HDL is likely discoidal.

The CERM are spherical particles with sizes (r ˜250 nm) and NL composition (˜80%; Table 2) comparable to those of chylomicrons, the largest plasma lipoproteins (Havel et al., 1980). Unlike chylomicrons, CERM is CE-rich making it the largest known CE-containing lipoprotein formed by a physiological reaction. In contrast to neo HDL, which are derived from a single HDL particle, many HDL contribute CE to CERM; these large particles (r=150-250 nm) have calculated volumes V˜(1 60)×107 nm³ so that each CERM contains the CE equivalent of >100,000 HDL particles. Intact rSOF is only found in the CERM (FIG. 2G). In contrast, an immunoreactive band with a mass of 50 kDa may be a fragment formed by an HDL-associated protease.

Thermodynamics of Opacification: The thermodynamics of the rSOF-mediated delipidation of HDL can be rationalized in the context of the principle-of-opposing-forces model of Tanford (Tanford, 1980). In the absence of amphiphiles, NL in water form a single phase that is stabilized by hydrophobic forces. In the presence of amphiphiles such as PL and to a lesser extent apos, a second force comes into play, i.e., the tendency of amphiphilic components to associate in ways that bury their hydrophobic surfaces but leave their polar or charged moieties available for high energy solvation by water. For PL, the hydrophobic surface is defined by their acyl chains and the solvation site is their zwitterionic headgroups; for apos the hydrophobic surface is the non polar face of their amphipathic helices and the polar sites are on the opposing helical surfaces that contain polar and charged amino acid residues. The balance of these two forces, determined by the physical properties of the reactant components, their relative abundance, and the attendant mechanisms, which may follow different reaction coordinates, determines the product profile, which lies somewhere between total phase separation and total homogeneity.

Given that opacification is spontaneous, the free energy of the reactants must be higher than those of the products, which must be stabilized by more favorable intermolecular forces. rSOF transfers most of the lipid components of HDL to the CERM leaving the balance of the lipid components with neo HDL. The exception, LF-apo A-I, is notable because it is the least lipophilic of all the components of HDL so that in the competition for the limited amount of PL in CERM and neo HDL, some apo A-I is excluded from both macromolecular species. Thus, one of the effects of rSOF is to remove low affinity apo A-I from HDL while leaving the higher affinity apo A-II, an effect that is emulated by chaotropic perturbation (Pownall et al., 2007). However, in the presence of adequate phospholipid, HDL-apo A-I is stabilized and apo A-I is not released (FIG. 8). Thus, phospholipid is essential to HDL stability and physiological activities that consume or transfer phospholipid LCAT, hepatic lipase, cholesteryl ester transfer protein, and phospholipid transfer protein would destabilize HDL-apo A-I (Rao et al. 1997; Lusa et al., 1996; Silver et al. 1990; Liang et al., 1996; Rye et al., 1997). The greater lability of apo A-I can also be inferred from human studies showing that the fractional catabolic rate of apo A-I in normolipidemic and hypo α-lipoproteinemic patients is greater than that of apo A-II (Brinton et al., 1991), presumably through greater renal loss of LF-apo A-I (Glass et al., 1983).

The rSOF-mediated redistribution of apo E and rSOF is distinct from those of apos A-I and A-II. At low HDL concentration, apo E apo A-II heterodimers are associated with the large HDL subfractions, one of which contains only a trace of dimeric apo A-II but no apo A-I (FIG. 1F, fraction 31). Higher reactant concentrations reveal apo E as monomers, homodimers, and heterodimers with apo A-II (FIG. 2G). Relative to the small neo HDL, which is rich in homo and heterodimers, in large neo HDL (Fractions 22-26), apo E monomers and heterodimers predominate. Although chemical analyses showed little or no lipid in fractions 22-26 (FIG. 2), reaction of rSOF with HDL labeled with [³H]CE (FIG. 7B, insert), shows increasing CE in this elution range at longer incubation times. Thus, like apo A-II, apo E is always observed as a lipidated species. Within the CERM fraction, monomeric apo E predominates.

Apo A-I Self Association: Although a well known phenomenon (Brinton et al., 1991; Vitello and Scanu, 1976), the relevance of apo A-I self-association to a physiological context has never been established. A reaction is demonstrated here, in which concentration-dependent apo A-I self-association determines a product profile under physiological conditions. Data (FIGS. 2 and 4) show clear evidence of apo A-I oligomerization within the typical human plasma HDL-protein concentration range of ˜1.5-2 mg/mL (Havel et al. 1980). Moreover, the oligomeric state corresponds to the apo A-I tetramers that have been identified by hydrodynamic methods (Vitello and Scanu 1976).

Speciated HDL Opacification: Data showed that the neo HDL formed from the smaller HDL species are smaller than those formed from the largest HDL (FIG. 5B-F) and that CERM formation is a linear function of HDL-NL content (FIG. 5G). Interestingly, the amount of LF-apo A-I is constant across a range of HDL sizes whereas the amount of neo HDL formed increases with decreasing size of the starting HDL (FIG. 6B). These data are consistent with formation of one neo HDL of fixed composition from each HDL particle. At constant HDL-protein concentration, there are fewer large particles so fewer neo HDL form for the same amount of LF-apo A-I. Although rSOF transfers most of the CE to CERM, a small amount appears in neo HDL (FIG. 6D). This reflects the sparing solubility of CE in PL (Hamilton et al. 1983) and the PL-rich nature of neo HDL. Consistent with this, the percentage of CE in neo HDL decreases with decreasing size and PL content (Table 3) of the HDL from which it was derived. Moreover, the higher amount of TG than CE in neo HDL reflects its higher solubility in PL (Hamilton et al., 1983). Thus, rSOF removes CE from the HDL core but leaves a small fraction that is solubilized by the PL.

A Mechanistic Model for Opacification: rSOF is catalytic; without being consumed, 1 μg/mL (˜10 nM) rSOF opacifies a 1000 molar excess of HDL (1 mg/mL=10 μM) and in the process transfers a >40,000 molar excess of CE to CERM. The process by which rSOF catalyzes the opacification of a 400 to >3000 molar excess of HDL (FIG. 1 and FIG. 2) and for kinetics that are exponential with respect to CERM growth and LF-A-I release is highly efficient.

In one embodiment, SOF transfers one or more CE particles from one HDL to another that eventually grows to a CERM through successive transfer cycles.

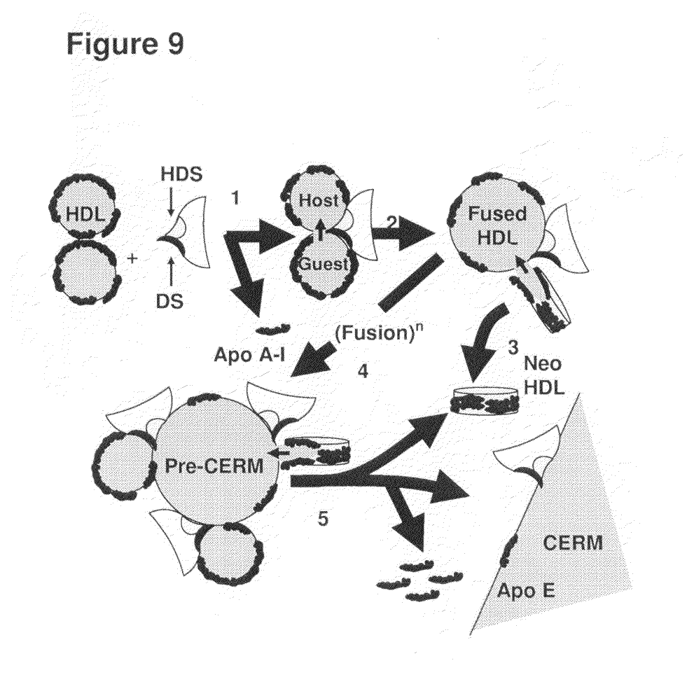

In another embodiment, rSOF is a heterodivalent fusogenic protein that binds to exposed CE surfaces that are formed by the desorption of apo A-I and recruits additional HDL-CE in multiple steps (FIG. 9). Step 1: rSOF binds to an HDL particle with its high affinity docking site (HDS) and displaces apo A-I, thereby forming the HDL host-rSOF complex that is destined to become a CERM. This could occur through rSOF insertion into the surface monolayer of HDL thereby raising the surface pressure and displacing the most weakly associated component, apo A-I, into the aqueous phase. Alternatively, spontaneous desorption of apo A-I into the aqueous phase could free up transient hydrophobic CE patches on the surface of HDL that are sites of rSOF-HDS insertion and docking. The surface association of rSOF is supported by data (FIG. 3) and previous studies (Courtney et al. 2006) showing that as the amount of rSOF increases, the size of CERM decreases, thereby providing a greater amount of total surface to accommodate an increasing number of rSOF molecules. A second “guest” HDL particle diffuses to the low affinity “delipidation site” (DS) of rSOF. Step 2: A transient guest-host complex forms and a continuous “stalk” joins the neutral lipid cores of the two HDL. Step 3: CE in the guest HDL particles transfers to and coalesces with those of the host HDL and the neo HDL is extruded into the aqueous phase; this step, CE partitioning into one compartment, would be expected to provide some of the free energy that drives the reaction. Step 4 comprises multiple cycles of host-guest interactions that ultimately form (Step 5) numerous neo HDL and CERM that contains rSOF and apo E as the only detectable proteins. Notably, the immunoblotting data (FIG. 2G) shows that the CE-containing particles eluting between the mature CERM and HDL (FIG. 7B) are more apo E-rich than the CERM. In a specific embodiment, this is due to the much greater total surface area and attendant higher surface content of PL, which mediates apo E binding.

Chemical kinetics: Kinetic data is shown in (FIG. 7). Under kinetic conditions, HDL (125 kDa @ 0.5 g/L; [HDL]=4×10⁻⁶ M) and rSOF (100 kDa @ ˜1 mg/L; [rSOF]=10⁻⁸ M) and the diffusion controlled rate constant kd=3×10¹¹ M⁻¹−min⁻¹. The initial step is the diffusion-controlled formation of the HDL-rSOF complex according to the following equation:

HDL+rSOF→HDL−rSOF Equation 1

This is followed by the multiple fusion reactions that form the growing CERM as given by

HDL−rSOF+HDL→CERM Equation 2

The concentration of the rSOF-HDL complex is equal to that of rSOF so that

rate=k_{d}[rSOF−HDL][HDL]=3×10¹¹M⁻¹−min⁻¹×4×10⁻⁶M×10⁻⁸M Equation 3

Given that first order kinetics were observed and that HDL is in great excess, Equation 3 gives a pseudo first order rate as ˜1×10⁻² min⁻¹, which corresponds well with the observed values of k_{CERM}=(3.2±0.008)×10⁻² and k_{AI}=(2.1±0.006)×10⁻² min⁻¹.

In one embodiment, interaction of rSOF with a surface CE patch is an important step in opacification, formation of LF-apo A-I determines specificity for HDL. VLDL and LDL are not appreciably opacified by rSOF (Courtney et al., 2006). Whereas, VLDL is relatively low in CE, LDL has a high CE content. However, neither contains apo A-I, the labile component of HDL (Mehta et al., 2003; Pownall, 2005; Sparks et al., 1992).

The desorption of apo A-I from HDL is a hallmark of its instability as revealed by both physico-chemical (Mehta et al., 2003; Pownall, 2005; Sparks et al., 1992; Pownall et al., 2007) and physiological perturbants (Rao et al., 1997; Lusa et al., 1996; Silver et al., 1990; Liang et al., 1996; Rye et al., 1997), some of which also produce CERM (Mehta et al., 2003; Pownall et al., 2007). However, not even chaotropic perturbation with 6 M guanidinium chloride produces as much of the CERM so that the rSOF reaction against HDL is unusual if not unique and unprecedented for a water-soluble protein. Two proteins have activities that share some characteristics with rSOF. One is microsomal transfer protein MTP-A, which catalyzes the coalescence of CE- and TG-rich particles during hepatic VLDL assembly (Wetterau et al., 1997). Within adipocytes, MTP-B, a splicing variant of the canonical MTP-A, appears to catalyze fusion of small TG-rich inclusions into large ones (Swift et al., 2005). The other protein, SR-BI, is an HDL receptor, that mediates net cellular internalization of HDL-lipids, especially CE. Similar to rSOF, SR-BI selectively removes CE from HDL at the cell surface while excluding apo A-I from net uptake (Acton et al., 1996; Glass et al., 1983).

Clinical Relevance: One of the remaining plasma lipoprotein risk factors for which current therapies are inadequate is low HDL cholesterol and its attendant dysregulated RCT. HDL opacification is a therapeutic modality for enhancing RCT because it rapidly transfers HDL-CE to a particle that contains apo E, a ligand for the hepatic LDL receptor, which could remove large amounts of HDL-derived CE. At the same time, neo HDL, which is CE-poor, is available to initiate additional cycles of cellular cholesterol efflux, esterification, opacification, and removal. Studies in mice in which SR-BI has been ablated or over expressed suggest that more efficient RCT due to increased SR-BI expression is associated with low HDL-C and reduced atherosclerosis (Varban et al., 1998; Kozarsky et al., 1997; Trigatti et al., 1999; Braun et al., 2002; Covey et al., 2003; Arai et al., 1999; Ueda et al., 2000). In one embodiment, improvement of multiple steps in RCT by rSOF in cellular and animal models of atherosclerosis would provide therapeutically appropriate opacification methods.

### Example 10

### Overview of Examples 12-15

Human plasma high density lipoproteins (HDL), the primary vehicle for reverse cholesterol transport, are the target of serum opacity factor (SOF), a plasma clouding factor that is secreted by Streptococcus pyogenes. HDL comprise a core of neutral lipids—cholesteryl esters and small amounts of triglyceride—surrounded by a surface monolayer of cholesterol, phospholipids, and specialized proteins—apolipoproteins (apos) A-I and A-II. HDL is an unstable particle residing in a kinetic trap from which it can escape via chaotropic, detergent or thermal perturbation. Recombinant (r) SOF catalyzes the transfer of nearly all neutral lipids of ˜100,000 HDL particles (D ˜7 nm) into a single, large cholesteryl ester-rich microemulsion (CERM; r>100 nm) leaving a new HDL-like particle-neo HDL (D ˜2 nm) while releasing ˜50% of the apo A-I in the lipid-free (LF) form. CERM formation and apo A-I release have similar kinetics suggesting parallel or rapid sequential steps. An embodiemnt of opacification by complementary physico-chemical methods is shown here. According to size exclusion chromatography, HDL containing non labile apo A-I resists rSOF-mediated opacification. According to kinetic cryo electron microscopy, rSOF (10 nM) catalyzes the conversion of HDL (4 μM) to neo HDL via a step-wise mechanism in which intermediate size particles are seen. Using similar conditions, kinetic turbidimetry revealed opacification as a rising exponential reaction with a rate constant k=4.4±0.004)×10⁻² min⁻¹. Analysis of the data using transition state calculated gave respective enthalpy, entropy and free energy of activation of ΔH^{‡}=73.9 kJ/mol, ΔS^{‡}=−66.87 J/° K, and G^{‡}=94.6 kJ/mol. The free energy of activation for opacification is nearly identical to that for the displacement of apo A-I from HDL by guanidine hydrochloride. In one embodiment, apo A-I lability is required for HDL opacification and that LF apo A-I desorption is the rate-limiting step.

### Example 11

### Materials and Methods for Examples 12-15

Materials: HDL was isolated according to its density by sequential flotation of human plasma obtained (Gillard et al., 2007); the HDL were further purified by SEC and for some tests HDL were subfractionated according to size by SEC using two Superose HR 6 columns (GE Healthcare, Piscataway, N.J.) in tandem (Gillard et al., 2007); fractions from multiple injections (0.5 mL) were pooled as needed for kinetic analysis. “Ultrastable” HDL (Pownall et al., 2007; Oram et al., 2000; Acton et al., 1996) was prepared by saturating HDL with guanidinium chloride (Gdm-Cl), warming to 40° C. for one hour, and stiffing at room temperature for 24 hours. After exhaustive dialysis against TBS, the density was adjusted to 1.21 g/mL by the addition of KBr and centrifuged at 40,000 rpm (Beckman Ti 50.2 rotor) for 24 hours. The apo A-II-rich HDL, i.e., ultrastable HDL, was collected from the top of the tube by pipette. A recombinant polyhistidine-tagged, truncated form of sof2, rSOF, encoding amino acids 38-843 was cloned and expressed in Escherichia coli (rSOF) and purified by metal affinity chromatography as described previously (Courtney et al., 1999).

Kinetic Cryo EM: Opacification was initiated by combining rSOF and HDL, 1 μg/mL (˜10 nM) and 0.5 mg/mL (˜4 μM) respectively, at 37° C. Aliquots were removed at 0 (no rSOF), 16, 45 and 94 min for vitrification, a rapid freezing process that spatially fixes particles in their native solution conformation without a chemical fixative. Vitrification was performed in liquid ethane using standard procedures on a Vitrobot (FEI, Inc.) vitrification robot. Frozen specimens were stored in liquid nitrogen until imaging on a JEOL 2010F electron microscope equipped with a field emission gun and JEOL semi-automated automation software (FasTEM). The microscope was operated at a specimen temperature of 97 K with an acceleration voltage of 200 keV. Images were recorded at a magnification of 50,000 with a GATAN 4k×4k CCD camera (Gatan, Pleasanton Calif.) at a total dose of 18 electron/A².

Images of individual HDL particles were selected at 0, 16, 45 and 94 min (5,000, 33,000, 8,000 and 35,000 particles respectively). The particle images were classified and averaged using an iterative alignment procedure in the EMAN (Ludtke et al., 1999) software package, which produces a set of characteristic views from a heterogeneous particle population. The linear dimension of each HDL class-average was measured and weighted by the number of particles contributing to that class. Averages were then integrated and interpolated yielding size distributions for each time point.

Kinetic Turbidimetry: The rates of rSOF-mediated opacification of HDL were measured as a function of temperature by kinetic turbidimetry which monitors light scattering produced by the appearance of the very large (>100 nm) CERM. After thermal preequilibration of HDL (0.8 mg/mL) at each temperature, rSOF (1 μg/mL) was added and the increase in right angle scattering light intensity at 325 nm was measured as a function of time on a Jobin Yvon Fluorolog. The intensity vs. time data were fitted to the growing exponential function, Iₜ=I₀+a(1−e^{-kt}), where I₀ is the initial scattering intensity, Iₜ is the intensity as a function of time (t), a is a pre exponential instrumental factor and k is the rate constant. Using transition state theory, the respective enthalpy (ΔH^{‡}) and entropy (ΔS^{‡}) of formation of the activated state were determined as ΔH^{‡}=Eₐ−RT, ΔS^{‡}=2.303 R log NhX/RT where R is the gas constant, T is the absolute temperature, k is the reaction rate constant, N is Avogadro's number, h is Planck's constant, and X=k/(exp−ΔH^{‡}/RT). The free energy of activation (ΔG^{‡}) was calculated as ΔG^{‡}, H^{‡}−TΔS^{‡}.

### Example 12

### Kinetic Cryo EM

rSOF catalyzes the disproportionation of HDL into small lipid-protein particles, neo HDL, and large lipid-protein particles, CERM, with the concomitant release of LF-apo A-I. Cryo electron microscopy was used to follow the rate of neo HDL formation. These studies revealed that addition of rSOF to HDL catalyzes profound changes in HDL structure.

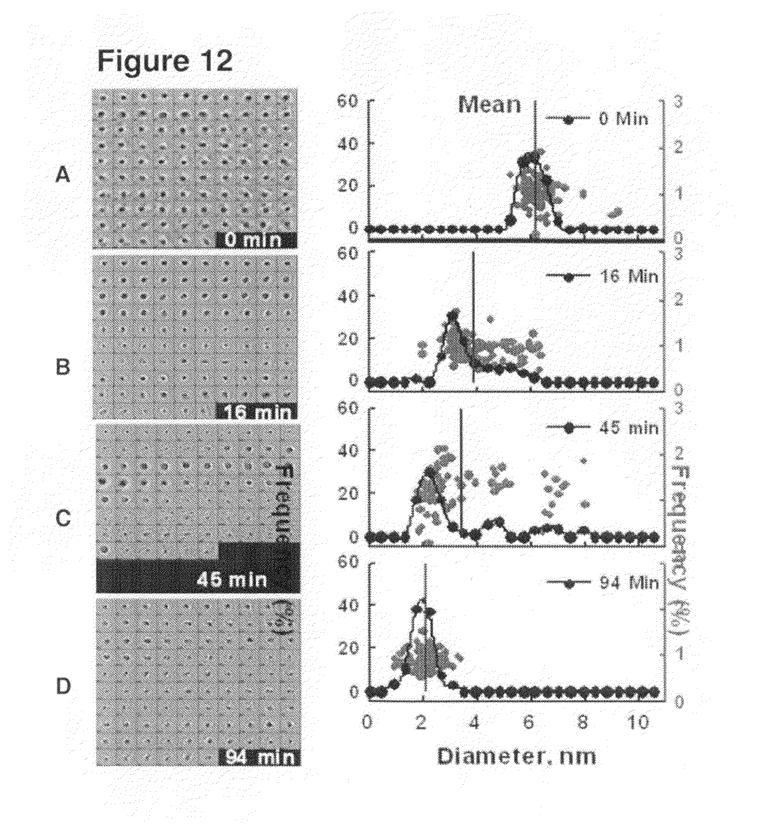

FIG. 12 provides views of HDL particles before and at various times following addition of rSOF. Samples were quick-frozen at various times after mixing and viewed on a JEOL 2010F electron cryo microscope. Images of individual particles (5000, 33000, 8000 and 35000 particles respectively) were selected at 0, 16, 45 and 94 min, classified, and iteratively averaged using EMAN software. At 0 min, most particles are ˜6.5 nm; at 94 min, the majority are ˜2 nm.

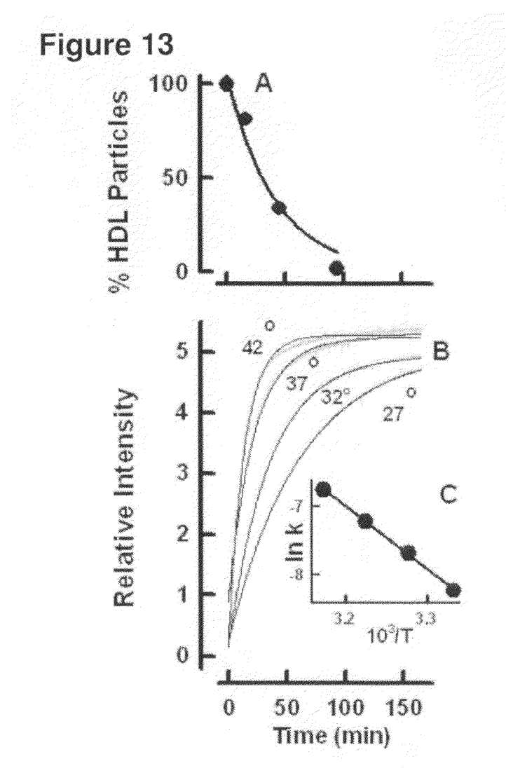

Control HDL comprise particles with d=6.53±0.18 nm (FIG. 12A). After addition of rSOF at 37° C., the HDL exhibit a gradual but profound transition from large particles at 0 min to small particles at 94 min, with particles of intermediate size being observed at 16 and 45 min. Notably, at 16 min few particles in the size range of HDL remain and only a small number in the range of new HDL have appeared (Circled in FIG. 12B). At 94 min, the large HDL seen at t=0 min, have been replaced by particles with d=1.98±0.05 nm. These sizes correspond to an rSOF-mediated reduction in particle volume from 167 to 4 nm³ (−95%). According to the disappearance of the ˜6.5 nm particles, the reaction is essentially complete at 94 min (FIG. 12D). Analysis of the rate data showed that rSOF-medated conversion of HDL to neo HDL was first-order with a rate constant, k=(2.42+0.54)×10⁻² min⁻¹ (FIG. 13A; r²>0.96).

### Example 13

### Kinetic Turbidimitry

Opacification of plasma is a hallmark of the reaction catalyzed by SOF. Thus, light scattering by the CERM particles, which are very large (>100 nm), provided another way—kinetic turbidimetry—to follow reaction kinetics in real time. According to kinetic turbidimetry (FIG. 13B), the rate of CERM formation at 37° C. is k=(4.4±0.004)×10⁻² min⁻¹, a value that compares well with the rate observed by kinetic cryo EM. The rates of opacification of HDL were temperature dependent and increased from 2.7 to 7.3 min⁻¹ between 300 and 310° K (FIG. 13B). The activation energy for CERM formation determined from its temperature dependence according to Arrhenius was 76.5 kJ/mol (FIG. 3B, FIG. 3C). On the basis of the rate constant at 37° C. and the activation energy, the thermodynamics of the transition state for CERM formation were calculated using transition state theory, which gave ΔH^{‡}=73.9 kJ/mol, ΔS^{‡}=−66.87 J/° K, and G^{‡}=94.6 kJ/mol. Thus, most (˜80%) of the free energy of activation for opacification enthalpically determined with the entropic component contributing the remaining ˜20% (Table 4, below).

TABLE 4
TRANSITION STATE PARAMETERS FOR HDL
OPACIFICATION BY RSOF

| | k, min⁻¹ | Eₐ, kJ | ΔH‡, kJ | −TΔS^{‡}, kJ | ΔG^{‡}, kJ |
|---|---|---|---|---|---|
| CP | 0.0032 | 110 | 107.5 | 11.6 | 95.9 |
| rSOF* | 0.044 | 76.5 | 73.9 | 20.7 | 94.6 |

*From data of FIG. 13B and FIG. 13C.

### Example 14

### Ultrastable HDL Resists Opacification

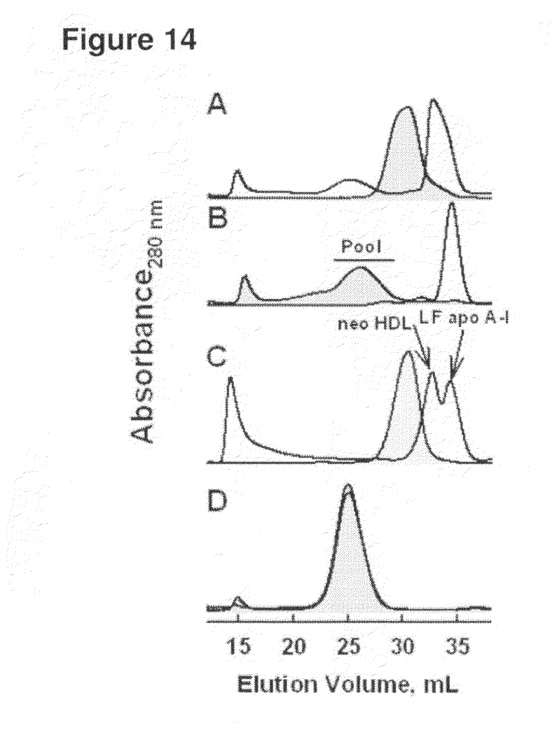

Chaotropic perturbation of HDL with Gdm-Cl releases a labile apo A-I fraction leaving an “ultrastable” apo A-II-rich particle (Mehta et al., 2003) from which additional apo A-I cannot be displaced by 6 M Gdm-Cl (Pownall et al., 2007). Saturating concentrations of Gdm-Cl (˜7 M) were incubated with HDL at 40 and 25° C. for 1 and 24 hours respectively and isolated two major fractions—ultrastable HDL and LF-apo A-I (FIG. 14A). Ultrastable HDL was isolated by flotation (FIG. 14B), and the effects of rSOF on HDL and ultrastable HDL were compared by SEC. As previously observed, rSOF converts HDL into three fractions—CERM, neo HDL, and LF apo A-I (FIG. 14C). In contrast, the rSOF had no effect of the SEC profile of ultrastable HDL (FIG. 14D). Thus, ultrastable HDL is highly resistant to opacification and does not release LF apo A-I in response to rSOF treatment.

### Example 15

### Effect of HDL Size on Opacification Kinetics

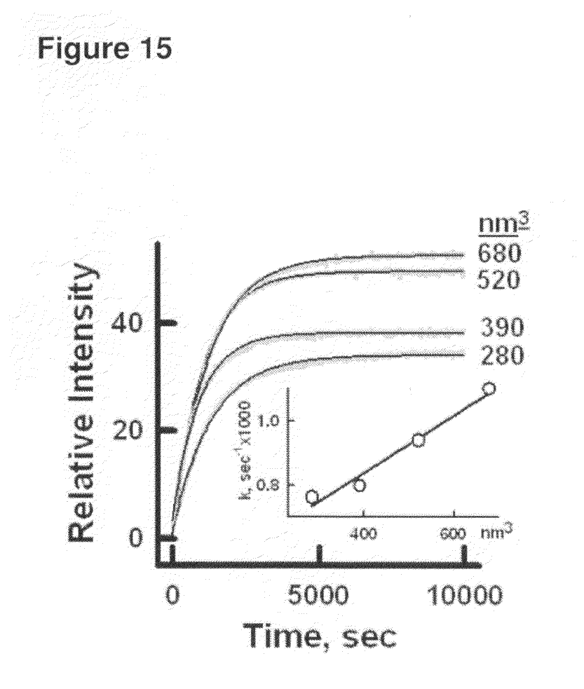

The rates of opacification of HDL subfractions isolated by SEC were compared by kinetic turbidimetry (FIG. 15). These data revealed a linear relationship between HDL particle size and opacification rate with opacification of the largest subfraction being slightly faster (+45%) than that of the smallest one. The rate constant for opacification increased linearly with HDL particle mass (FIG. 15, insert).

### Example 16

### Significance of Examples 12-15

Kinetic Cryo EM: On the basis of SEC data, one emobdiment of SOF action is as a heterodivalent protein that binds and delipidates HDL particles in one concerted step that transfers the HDL-CE to a growing CERM. However, SEC does not have the size-discriminating power of cryo EM, which in the present study provides “snap shots” of the SOF reaction at various reaction time points. The data show a relatively narrow distribution of HDL and neo HDL sizes at 0 and 94 min with virtually no particles of intermediate size. In contrast, at 16 min, few of the particles remain within the HDL size range and yet very few have been fully converted to particles with the dimensions of neo HDL; at 16 min, the sizes of most particles are between those of HDL and neo HDL. In one embodiment SOF-mediated delipidation of HDL in which some if not most HDL are only partially delipidated by SOF and then released as intermediates that are seen at 16 and 45 min. These intermediates then reassociate with the growing CERM, seen by turbidimetry (FIG. 13), via SOF, which completes the delipidation process.

HDL-Size Dependence of Opacification: The previous data showed that the kinetics of opacification and the release of apo A-I were similar (Gillard et al., 2007). Other studies have shown that the rate of spontaneous lipid desorption increased with decreasing lipoprotein size (Massey et al., 1984). This is in accordance with the Kelvin equation, which states that the rate of desorption from particle surfaces is an inverse function of particle radius. The kinetic data from Examples 12-15 show the opposite effect; opacification rates are a positive function of HDL size so that simple spontaneous LF apo A-I desorption is not likely involved in the rate-limiting step.

Apo A-I Lability is Central to HDL Opacification: Release of LF apo A-I and CERM formation have similar kinetics (Gillard et al., 2007) suggesting that these two steps are mechanistically linked. Three pieces of evidence suggest that this embodiment of opacification is dependent on the lability of apo A-I and that this step likely precedes CERM formation. First, LDL and VLDL, lipoproteins that do not contain apo A-I, are resistant to opacification (Courtney et al., 2006). Second, ultrastable HDL which contains non labile, i.e., nondissociable apo A-I but is similar to native HDL in most other respects including lipid composition (Courtney et al., 1999) is highly resistant to rSOF-mediated opacification (FIG. 4). Lastly, the transition state energetics of opacification were compared with that for the chaotropic perturbation of HDL with Gdm-Cl. Like opacification, chaotropic perturbation releases LF apo A-I (˜50% of total HDL protein); unlike opacification, chaotropic perturbation does not form neo HDL nor CERM (Courtney et al., 2006). Remarkably, the free energy of activation for opacification, 94.6 kJ, is virtually identical to that of the chaotropic perturbation of HDL (95.9 kJ; Table 4). Given that the common characteristics of opacification and chaotropic perturbation of HDL are formation of LF apo A-I and similar free energies of activation, an embodiment of apo A-I desorption is the rate-limiting step in HDL opacification. Moreover, another embodiment is that apo A-I is necessary but not sufficient for opacification, and that some of the HDL-apo A-I must be labile; given that all HDL is converted to rHDL, all HDL particles contain at least one molecule of labile apo A-I.

Physiologic Insights: HDL is an unstable particle that resides in a kinetic trap from which it can escape by thermal, chaotropic and detergent perturbations, all of which release LF apo A-I (Mehta et al., 2003; Pownall, 2005; Sparks et al., 1992; Pownall et al., 2007). This instability, which is elicited in part by HDL-remodeling proteins—cholesteryl ester and phospholipid transfer proteins, lecithin:cholesterol acyltransferase—is part of what make HDL unique (Lusa et al., 1996; Silver et al., 1990; Liang et al., 1996; Rye et al., 1997). As posited by Curtiss et al. (Curtiss et al., 2006) some these activities are likely important in the formation of LF apo A-I that is needed to remove free cholesterol from macrophages. HDL instability is also likely involved in the selective uptake of CE by SR-BI-expressing cells, a process in which CE but not apo A-I is internalized. In one embodiment of the invention, the products formed by rSOF activity may have therapeutic value. rSOF forms LF apo A-I, which interacts with ABACA1 thereby promoting cholesterol efflux via a microsolubilization mechanism; neo HDL, which is free cholesterol-poor, is expected to be a better acceptor of cholesterol via ABCG1; lastly, in another embodiment, the CERM is a vehicle for improved hepatic CE removal.

### Example 17

### Materials and Methods for Examples 18-21

Materials: DPH, TMA-DPH, Laurdan, and Patman were purchased from Molecular Probes (Grand Junction, Oreg.). HDL was isolated according to its density by sequential flotation of human plasma obtained (Schumaker and Puppione, 1986); fractions from multiple injections (0.5 mL) were pooled as needed. TBS (100 mM NaCl, 0.01% NaN₃, 0.01% EDTA, and 10 mM Tris) was used throughout. A polyhistidine-tagged, truncated form of sof2 encoding amino acids 38-843 was cloned, expressed in Escherichia coli, and purified by metal affinity chromatography as described (Courtney et al., 1999, 2006).

Methods: Apolipoprotein compositions were determined by SDS PAGE using 15% Tris-Glycine Ready Gels (BioRad). Particle charge was measured as previously described (Gaubatz et al., 2007) by electrophoresis in 0.79% agarose (90 mM Tris, 80 mM borate [pH 8.2]). HDL and neo HDL (5 g protein in <20 L) were loaded onto the gels and electrophoresis was performed at 4° C. at 90 volts for 90 minutes. Electrophosphoretic bands were visualized with Pierce GelCode Blue stain reagent, destained, and recorded by photography. The compositions of HDL and the products of opacification were determined using commercial kits for protein (BioRad DC Protein Assay) and for cholesterol, cholesteryl ester, triglyceride, and PC (Wako Chemicals USA, Inc. Richmond, Va.).

HDL Opacification and Isolation of Neo HDL and CERM: Both HDL and the partially purified neo HDL were purified by SEC as previously described (Pownall, 2005, Gillard et al., 2007). HDL (100 mg) was incubated with rSOF (16 rig) in 5.7 mL TBS for 24 h at 37 EC after which the sample was adjusted to d=1.063 g/mL by the addition of KBr, overlaid with 3 mL TBS (d=1.006 g/mL), and centrifuged at 40,000 rpm in a Beckman Ti 50.2 rotor for 18 hours. The CERM (˜2 mL) was removed from the top by pipetting. The infranatent was siphoned from the bottom of the tube into 2 mL fractions, which were analyzed by SEC. Those richest in neo HDL were pooled and fractionated in a gradient of KBr in TBS (d=1.21 to 1.12 g/mL; 48 h @ 40,000 rpm, Beckman SW 50.2). The supernatant containing neo HDL was removed from the top of the tube by pipette.

Lipid Analysis: Lipid compositions were determined by high performance thin layer chromatography (HP-TLC) as previously described (Gaubatz et al., 2007). HDL was split into two equal fractions (45 mg/2 mL). One was untreated and the other was incubated with rSOF (5 g) for 24 h; SEC showed quantitative conversion of HDL to CERM and neo HDL. The CERM was separated from the neo HDL by floatation in TBS containing KBr at d=1.063 g/mL for 18 hours at 32,000 rmp (Beckman SW 40.1). The CERM, which appeared as a compact pad at the top of the tube, was removed by pipette. Another ˜8 mL was removed by aspiration and the bottom fraction containing neo HDL was collected. The CERM, neo HDL, and HDL were dialyzed vs. ammonium bicarbonate, lyophilized, and extracted twice with two parts chloroform plus one part methanol. The solvent was reduced to dryness under a stream of nitrogen and the residue dissolved in ˜0.5 mL chloroform. Aliquots (100 μL) of CERM-, neo HDL-, and HDL-lipids were applied to plates containing a thin layer of silica and the lipids eluted using a two solvent system that separates polar and non polar lipids (Gaubatz et al., 2007). The lipids were quantified by staining with primulin and measuring the lipid-associated fluorescence by phosphorimaging (GE Healthcare Storm 840). The identity of the lipids was confirmed by comparing their elution positions with authentic lipid standards. The lipid compositions are expressed as a percent of total composition. Compositions were compared by Student's t-test with a p<0.05 being considered significant.

Fluorescence Spectroscopy: Several well characterized fluorescent probes of the properties of lipids that were previously used to characterize native and model human plasma lipoproteins (Massey and Pownall, 1998) were used to compare HDL, neo HDL, and CERM. The polarization of a fluorescent probe increases with increasing environmental microviscosity. The fluorescence polarization of DPH, which partitions equally between surface and core lipids of plasma lipoproteins, reflects average microviscosity of the surface and core; the fluorescence polarization of TMA-DPH senses the microviscosity of the acyl chain and the headgroup regions of surface phospholipids (Prendergast et al., 1981, Massey et al., 1985a); Patman and Laurdan are fluorescent probes of interfacial polarity (Parasassi et al., 1994, Massey et al., 1985b). The probes in ethanol were added to purified HDL, neo HDL, and CERM with vortexing at the rate of ˜1 probe molecule/500 phospholipid molecules.

Fluorescence measurements were performed on a Jobin Yvon Spex Fluorolog-3 FL3-22 spectrofluorometer (Edison, N.J.), equipped with Glan-Thompson polarizing prisms as previously described (Massey and Pownall, 2005, 2006); polarization (P) was corrected for monochromator effects on polarized light. Using a Peltier controller, the sample temperature was increased in 1° C. increments and equilibrated for 1 min after which the polarization was recorded. Slopes of P vs. T were determined by linear regression analysis of the data (Sigma Plot 8.0). The excitation and emission settings are given in the Figure legends. The general polarization (G. P.) of Laurdan was calculated from the intensities of the short and long wavelength peaks in its fluorescence spectra according to G. P.=(I₄₃₀−I₄₈₀)/(I₄₃₀+I₄₈₀) where I₄₃₀ and I₄₈₀ respectively are the fluorescence intensities at 430 and 480 nm. The G. P. of Patman was calculated similarly and based on the intensities for short and long wavelength peaks, which were different for HDL, neo HDL, and CERM; these were respectively 441 and 464 nm, 451 and 489 nm, and 419 and 462 nm.

### Example 18

### Compositions of HDL, Neo HDL, and CERM

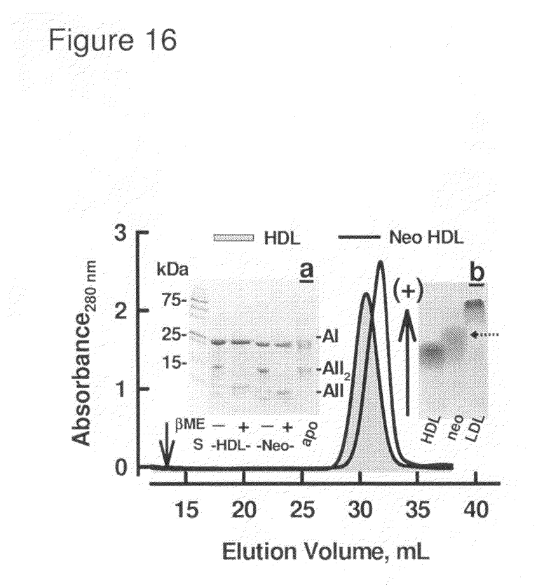

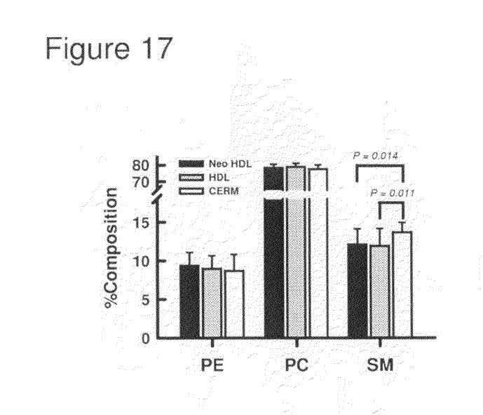

According to SEC analysis, the HDL and neo HDL were homogeneous (FIG. 16). Neo HDL is more apo A-II-rich than HDL (FIG. 16, insert a); their respective Stokes' radii were 9.7 and 10.8 nm. According to agarose gel electrophoresis (FIG. 16, insert b), neo HDL has pre β mobility. The compositions of neo HDL and CERM were distinct from those of HDL (Table 5). Neo HDL contained less CE and its calculated mol % FC compared to PL was less than half that of HDL. In contrast, the CERM were very CE-rich and contained very little PL or protein. Moreover, the mol % FC in CERM was ˜3 times that of HDL; the ratios of the core to surface lipid masses, exclusive of FC, increase in the order neo HDL<HDL<<CERM. rSOF produced a subtler but still significant segregation of phospholipid species with sphingomyelin (SM) preferentially transferring to CERM (FIG. 17). The PC contents of HDL, neo HDL and CERM were similar whereas there was a non significant enrichment of neo HDL with phosphatidylethanolamine.

TABLE 5
CHEMICAL COMPOSITIONS (%) OF HDL, NEO HDL, AND CERM

| Analyte | HDL | Neo HDL | CERM |
|---|---|---|---|
| PL | 25.5 ± 1.3 | 43.9 ± 0.8 | 4.3 ± 0.01 |
| FC | 2.2 ± 0.1 | 1.6 ± 0.06 | 1.8 ± 0.13 |
| CE | 14.5 ± 1.6 | 2.2 ± 0.16 | 78.1 ± 2.7 |
| TG | 4.8 ± 0.5 | 4.2 ± 0.39 | 6.9 ± 0.8 |
| Pro | 53.0 ± 6.8 | 48.0 ± 4.2 | 8.8 ± 0.8 |
| Mol % (FC)^{a} | 14.5 | 6.7 | 45 |
| Core/Surface^{b} | 0.76 | 0.15 | 19.8 |

^{a}Mol % (FC) = 100 × [% FC/386]/[% FC/386 + % PL/760]; 387 and 760 are the molecular masses of FC and PL.
^{b}Core/Surface = (CE + TG)mass/PL mass.

### Example 19

### Intrinsic Fluorescence Spectra

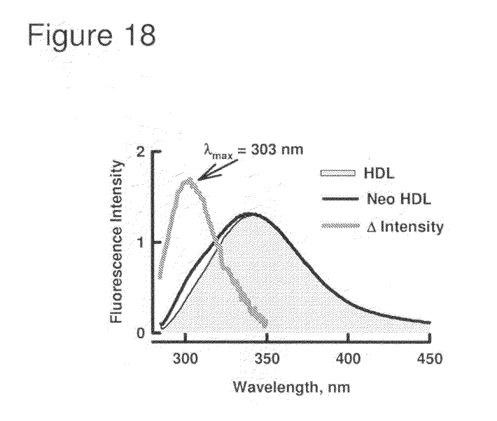

Apos A-I and A-II contain two near ultraviolet chromophores-tyrosine and tryptophan, which have respective molar extinction coefficients 1280 and 5690 cm-M⁻¹ at 280 nm (Edeldhoch, 1967). Unlike apo A-I, which contains four tryptophan and six tyrosine residues, apo A-II contains no tryptophan but has eight tyrosine residues (Brewer et al., 1986). Thus, neo HDL, which is apo A-II rich (FIG. 16, insert a), might have a fluorescence spectrum that is distinct from that of HDL. As shown in FIG. 18, both HDL and neo HDL exhibit fluorescence maxima at 341 nm. However, the fluorescence spectrum of neo HDL is distinguished by a shoulder on the short wavelength side of its spectrum, and the difference between the normalized fluorescence spectra of HDL and neo HDL reveals an underlying spectrum with a peak at 303 nm.

### Example 20

### Lipid Micro Viscosities of HDL, CERM, and Neo HDL are Distinct

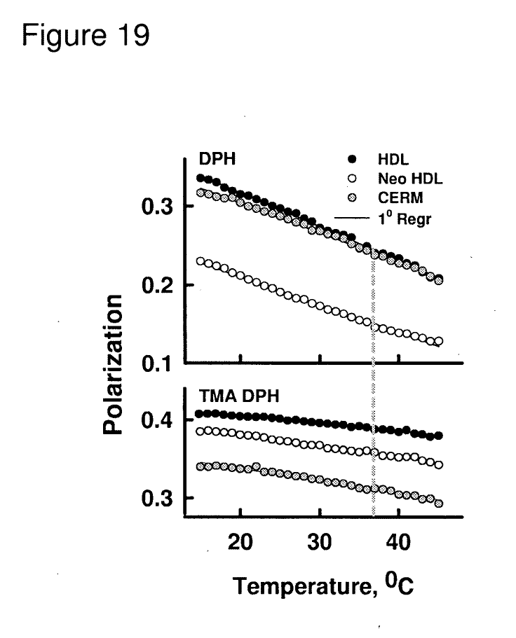

The microviscosities of the products of HDL opacification were determined as a function of temperature according to the polarization of the fluorescence of the lipophilic probe, DPH, which diffuses equally between surface and core compartments and provides an average microviscosity of the surface and core lipids, and TMA-DPH, which senses changes in the microviscosity of the surface monolayer that surrounds lipoproteins. According to DPH fluorescence polarization, the total microviscosities of HDL and CERM and their temperature dependence are similar and much higher than that of neo HDL (FIG. 19; Table 6). In contrast, the polarization of TMA-DPH shows that the microviscosities of the lipoprotein surfaces decrease in the order HDL>neo HDL>CERM. The temperature dependence of the surface microviscosity as assessed from fluorescence TMA-DPH polarization is similar for all three particles (FIG. 19, Table 6).

TABLE 6
MICROVISCOSITY PARAMETERS FOR
HDL, NEO HDL, AND CERM

| | DPH | | TMA DPH | |
|---|---|---|---|---|
| | P (37 EC) | (P/EC) × 10³ | P (37 EC) | (P/EC) × 10³ |
| HDL | 0.24 | −4.3* | 0.39 | −0.97* |
| Neo HDL | 0.15 | −3.5* | 0.36 | −1.4* |
| CERM | 0.24 | −3.7* | 0.31 | −1.5* |

*Calculated by a linear regression fit of the data; r² > 0.97.

### Example 21

### Surface Polarity

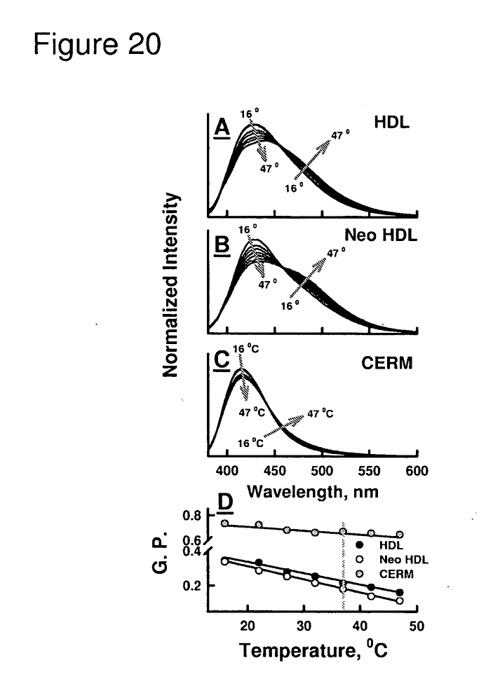

The surface polarities of HDL, neo HDL, and CERM were assessed from the G. P. of Laurdan and Patman, which were calculated from their respective fluorescence spectra. Both Laurdan and Patman exhibit two spectral maxima, the relative magnitudes of which respond to the polarity of the probe environment; increasing G.P. corresponds to the decreasing polarity of the probe environment. G. P. of Laurdan is also sensitive to changes in the physical state of phospholipid in the glycerol region of the phospholipid molecule (Parasassi et al., 1991); during thermal transition of phospholipids from the gel to the liquid crystalline phase, the polarity of the interfacial region is increased by increased hydration, an effect that is reversed in part by the addition of cholesterol (Parasassi et al., 1990, 1991, Massey, 2001). The G. P. (Laurdan) revealed differences between the surface properties of HDL, neo HDL, and CERM. At 37 EC, the G. P. (Laurdan) for HDL and neo HDL were similar indicating similar polarities in the glycerol backbone region (FIG. 20A, FIG. 20 B). The slopes of the curves for the temperature dependence of G. P. were also similar for HDL and neo HDL (FIG. 20D; Table 7). The much higher G. P. (Laurdan) of CERM (FIG. 20C) is consistent with an environment in the glycerol backbone region that is much less polar than those of HDL and neo HDL. The change in G.P. (Laurdan) with temperature was much smaller than that of HDL and neo HDL (FIG. 20D; Table 7).

TABLE 7
G.P. PARAMETERS FOR HDL, NEO HDL, AND CERM

| | Laurdan | | Patman | |
|---|---|---|---|---|
| | G.P. | | G.P. | |
| | (37 EC) | (G.P./EC) × 10³ | (37 EC) × 10 | (G.P./EC) × 10³ |
| HDL | 0.2133 | −6.32* | −0.515 | −4.68* |
| Neo HDL | 0.1794 | −7.33* | −0.965 | −9.39* |
| CERM | 0.6712 | −2.88** | −0.123 | −14.3* |

*r² > 0.98;
**r² > 0.87

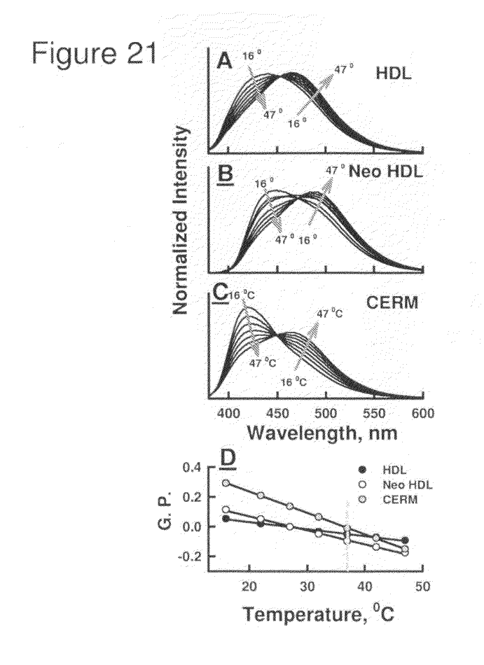

Similar G. P. data were collected for Patman whose structure contains a positively charged quaternary amino group that confines it to a more hydrated region of the particles that is closer to the lipid-water interface (FIG. 21; Table 7). Although the G.P. (Patman) and its temperature dependence simulated those of Laurdan, the CERM results were quite distinct. Like G. P. (Laurdan), G. P. (Patman) was similar for HDL and neo HDL at all temperatures and distinct from that of CERM except at 37 EC where G. P. (Patman) was nearly the same for HDL, neo HDL, and CERM (FIG. 21). This observation was a consequence of the much more profound temperature dependence of G. P. (Patman) vs. Laurdan for CERM (FIG. 20C, FIG. 21C, and Table 7).

### Example 22

### Significance of Examples 18-21

Plasma lipoproteins comprise a central neutral lipid core of CE and TG surrounded by a surface monolayer of phospholipids and apos. rSOF catalyzes the formation of two new particles from HDL, neo HDL and CERM, which are profoundly different from HDL with respect to both size and composition (Courtney et al., 2006, Gillard et al., 2007, Table 5). Neo HDL are smaller and contain less free cholesterol and neutral lipid than do HDL (FIG. 16; Table 5). According to the previous data, rSOF produces a profound segregation of lipid species with the fraction of a given HDL-lipid transferring to CERM increasing in the order PL<FC<TG<CE. HP-TLC analysis confirmed this finding (Table 5) that the more hydrophobic lipids preferentially associate with the CERM. rSOF also catalyzed a small, but significant segregation of SM, the most hydrophobic HDL-phospholipid. Thus, the mechanism for opacification must involve a sorting step that is based on lipid hydrophobicity.

Relative to HDL, neo HDL is rich in apo A-II (FIG. 16 insert a), which has no tryptophan but contains eight tryosine residues. Consequently, a shoulder due to tyrosine fluorescence can be discerned by difference spectroscopy. In contrast, the CERM are very large and heterogeneous with dimensions approaching 500 nm (Courtney et al., 2006, unpublished results).

DPH Polarization: Among model lipoproteins, cholesterol exerts a viscogenic effect that increases the polarization of fluorescence of embedded probes (Mantulin et al., 1981, Massey et al., 1985c). In contrast, the cholesterol content of neo HDL, HDL, and CERM was a poor predictor of total microviscosity as assessed by DPH fluorescence polarization. The mol % FC increased in the order, neo HDL<HDL<<CERM (Table 5), but the total microviscosity increased as neo HDL<HDL˜CERM. According to the calculated core-to-surface ratios (Table 5), neo HDL have little or no core, whereas HDL have nearly equal amounts of core and surface lipids. The discrepancy is particularly profound for CERM and HDL, which have similar total microviscosities, whereas the mol % FC for CERM is much higher (45%) than that of HDL (14.5%). This is likely due to the distribution of DPH into both the core and surface of the particles so that the DPH preferentially senses the core lipids which are in great excess of the surface lipids and are apparently highly fluid. In contrast, the viscogenic effects of cholesterol are seen when HDL (P=0.24; mol % FC=14.5%) and neo HDL (P=0.15; mol % FC=6.7%), which have very little core material, are compared. Although the major component of the CERM is CE, which exhibit thermal transitions between 20 and 40 EC, no discontinuities indicative of a transition were seen in the temperature dependence of the DPH fluorescence polarization in CERM or HDL and neo HDL.

TMA DPH Polarization: The major determinants of the surface microviscosities of lipid bilayers and lipoproteins are the phospholipid composition and the cholesterol content (Massey, 2001, Massey and Pownall, 2006). Although rSOF segregates phospholipids (FIG. 16) the magnitude of the segregation too small to substantively alter surface microviscosity. On the other hand, differences in the FC content of neo HDL, HDL, and CERM are expected to be reflected in the surface microviscosities. For example, according to TMA DPH polarization, the surface microviscosities of human lipoproteins increase in the order of their increasing mol % FC˜VLDL˜LDL<HDL₃ (Massey and Pownall, 1998). TMA-DPH fluorescence polarization reveals that the surface microviscosity of neo HDL is lower than that of HDL. This difference is no doubt due to the lower mol % FC in neo HDL. The surface microviscosity of CERM is even lower (P=0.31) than those of HDL (P=0.39) and neo HDL (P=0.36) despite having the highest mol % FC, 14.5, 6.7, and 45% respectively. It appears that the high mass of the CERM core also modulates the surface viscosity but in an indirect way. Whereas the surface microviscosities increase as CERM<neo HDL<HDL, mol % FC increases in the order neo HDL<HDL<<CERM, with CERM having by far the highest FC content but the lowest surface microviscosity according to TMA DPH fluorescence polarization. This discrepancy can be rationalized by observing that free cholesterol is soluble in neutral lipids such as CE and TG. Nuclear magnetic resonance studies show that about one third of ¹³C-labeled FC is in the particle core of human LDL, which based on composition (Havel et al., 1980) has a core to surface ratio of ˜2.2. This gives a Surface/Core partition coefficient K=[2/1]/[1/2.2]˜4. According to this partition coefficient and the Core/Surface ratio for CERM (Table 5), only ˜4% of the FC resides in the PL surface and 96% is in the neutral lipid core. Reduction of the core FC content by 96% gives a calculated mol %˜3, a value that is consistent with the low microviscosity that is observed according to TMA DPH fluorescence polarization.

The Physical Properties of Neo HDL Support Cellular Cholesterol Efflux: Their distinct composition, size, and pre β mobility (FIG. 16 insert b) suggest that neo HDL is similar to discoidal HDL formed via the interaction of apo A-I with ABCA1 (Duong et al., 2006). Comparison of the properties of HDL and neo HDL suggest that the latter would better support the first step in RCT-cholesterol efflux. First, PL are the essential FC-binding component of lipoproteins (Pownall, 2006, Fournier et al., 1996, 1997); the data show that relative to protein, neo HDL contain nearly twice as much PL as HDL. Second, as the FC content of rHDL increases, it becomes a poorer acceptor of net cellular cholesterol efflux from human skin fibroblasts, and at ˜15 mol % rHDL converts from acceptor to donor. Concurrently, fibroblast 3-hydroxy-3-methylglutaryl coenzyme A reductase declines and cellular CE formation via acyl CoA:cholesterol:acyltransferase increases (Picardo et al., 1986). HDL-FC is close to the mol % “switch” (Table 5). In contrast, neo HDL-FC is lower (Table 5) so that its capacity for additional FC is expected to be greater. Third, small particles, e.g., rHDL, are better acceptors of cholesterol than large particles such as single bilayer vesicles (Davidson et al., 1997), and according to the SEC data (FIG. 16), neo HDL is smaller than HDL. Although neo HDL, which has pre β mobility, is more electronegative than HDL, the effects of charge difference on FC efflux and other components of RCT are difficult to predict. HDL with pre β mobility is a preferred FC acceptor (Castro and Fielding, 1988), and increasing rHDL electronegativity by addition of phosphatidyl inositol potentiates efflux. However, no mechanistic link between particle charge and efflux has been established. Neo HDL is also expected to better support the second step in RCT, remodeling by LCAT. The lower microviscosity and cholesterol content of neo HDL is expected to make it a better substrate for LCAT, which preferentially esterifies FC in an environment of low microviscosity (Pownall, et al., 1985) and low cholesterol content (Simard et al., 1989).

Apo E is a minor component of HDL and following HDL opacification CERM contains apo E as its only apo (Gillard et al., 2007). This occurs in spite of the presence of other proteins, especially apo A-I, which is LF and in great excess. Given that apos associate with lipoprotein surfaces, preferential association with CERM must reflect distinctive surface qualities not present in neo HDL. Lipoprotein size is an important macromolecular determinant of apo E binding; as reflected in the CERM composition, apo E preferentially associates with large particles (Asztalos et al., 2007). However, the molecular basis for this and the specific lipid-protein interactions involved have not yet been identified. Surface microviscosity is not likely important; the microviscosity of neo HDL lies between those of HDL and CERM, both of which bind apo E (Table 6). Clues may be provided by the G.P. measurements. According to the G. P. of Patman and Laurdan, the surface polarization of CERM is much greater than those of HDL or neo HDL. What molecular determinant that is reflected in G. P. could support preferential apo E binding to CERM? Laurdan and Patman are sensitivity to the polarity and the molecular dynamics of the dipoles in their environment so that dipolar relaxation is reflected in large spectral shifts that are expressed in terms of G. P. Water molecules are the main solvent dipoles around Laurdan and Patman in lipid surfaces. In the absence of relaxation, GP values are high, indicating low water content at the interfacial region. Thus, CERM are distinguished from neo HDL by a lower interfacial polarity that would be expected to enhance associations with apos mediated by the hydrophobic effect. Neo HDL has a G. P. and hence an interfacial hydrophobicity that is lower than those of HDL and especially CERM. The absence of apo A-I in the CERM may be due to the interplay of two factors. First, apo E is more lipophilic than apo A-I, particularly with respect to association with large particles (Oran and Vaughan, 2006). Second, as a consequence the CERM surface is saturated with apo E. Thus, apo A-I is sterically excluded from the CERM by higher affinity binding of apo E. Apo E would be expected to target CERM to LDL-receptors, which, following rSOF treatment, could hepatically clear large amounts of plasma cholesterol.

Pharmacologic Potential: Accumulation of cholesterol in arterial macrophages produces an atherogenic state unless there is a mechanism for its disposal. That mechanism, RCT, comprises cellular cholesterol efflux to early forms of HDL in plasma where it is esterified by LCAT, and disposal of mature forms of HDL by the liver. Identifying new therapeutic strategies that enhance RCT is an important public health priority. It is unlikely that rSOF mediated opacification will be used therapeutically. However, given that the mechanism for opacification is known (Gillard et al., 2007) there is potential for identification of agents that catalyze the opacification that leads to LF apo A-I, neo HDL, and CERM. Neo HDL has a lower free cholesterol to phospholipid content than the HDL from which it was derived and is, as a consequence, a better acceptor for cholesterol efflux than HDL. In one embodiment, the CERM clears large quantities of cholesteryl esters via the hepatic LDL-receptor. Moreover, the LF apo A-I released by rSOF enhances RCT via interactions with the ABCA1 lipid transporter (Oram and Vaughan, 2006).

### Example 23

### Cellular Cholesterol Efflux to Neo HDL is Higher than that to HDL

THP-1 cells were labeled with [³H]cholesterol with intracellular esterification being inhibited by FR 179254. Various concentrations of neo HDL and the HDL from which it was formed were incubated with cells and the lipoprotein-associated radiolabel measured after 2.5 hr (FIG. 23). Whereas the maximum efflux to neo HDL was slightly greater than that to HDL (Vₘₐₓ=5.9±0.4 vs. 4.8±0.39%/hr), efflux to neo HDL was associated with a lower Kₘ (39±9 vs. 74±18 Φg/mL) so that the catalytic efficiency (E=Vₘₐₓ/Kₘ) was higher for neo HDL than for HDL (0.15 vs. 0.065%/hr/μg).

### Example 24

### Exemplary Studies

HDL Opacification is a Rational Therapeutic Pathway: As discussed previously, a recombinant (r) virulance determinant from S pyogenes (Cunningham, 2000; Courtney et al., 2006) serum opacity factor (rSOF), destabilizes and selectively delipidates human HDL by a mechanism that yields a cholesteryl ester-rich microemulsion (CERM), lipid-free (LF) apo A-I and a phospholipid-rich “neo HDL” (Gillard et al., 2007). In one embodiment of the invention, these products are clinical modalities for enhancing three steps in reverse cholesterol transport (RCT). In a specific embodiment, with its apo E and high CE content, CERM transfers large amounts of CE to the liver for disposal via the LDL receptor; and/or neo HDL is a better acceptor of cellular cholesterol than HDL (see below); and/or lipid-free apo A-I enhances efflux via ABCA1. The following molecular embodiments are tested.

Cholesterol efflux from peritoneal macrophages from WT, ABCA1-, and ABCG1-KO mice is measured. In one embodiment, macrophage-cholesterol efflux to neo HDL is higher than that to the HDL from which it was derived; and/or efflux via ABCG1 is higher than that via ABCA1. The rates of CE uptake from HDL with that of the products of the sequential actions of SOF and LCAT on HDL in hepatocytes from WT and SR-BI KO mice are compared. In one embodiment, hepatic CE uptake of LCAT-modified (neo HDL+cholesterol) is similar to that of HDL. The turnover of CERM-[3H]CE is measured and the tissue sites for CERM uptake is determined. In one embodiment, the liver is the major site of CERM disposal. Uptake and metabolism of HDL-CE with that of CERM-CE by hepatocytes from WT, LDL-receptor KO, and SR-BI KO mice are compared. In an embodiment of the invention, hepatic disposal of CERM-CE via the LDL-receptor is more efficient than that of HDL-CE via SR-BI. The transfer of peritoneal macrophage-cholesterol to plasma, liver and feces in mice treated with neo HDL with those treated with native HDL, rHDL, and control saline are compared. In one embodiment of the invention, Neo HDL is a better mediator of RCT than HDL. Lesion formation in apo E KO mice infused with native HDL, rHDL, or saline with those infused with neo HDL is also compared. In an embodiment of the invention, neo HDL reverses atherosclerosis.

### Example 25

### Exemplary Models

The following are exemplary models as to the working of HDL and therapeutic pathways that correspond to RCT.

RCT: Unlike liver, extrahepatic tissues synthesize but cannot degrade cholesterol. Thus, cholesterol accumulation within subendothelial-macrophages, a key cell type in atherogenesis, produces a lipotoxic, pathological state, unless there is a mechanism for removal and disposal; that mechanism is RCT, comprising (1) cellular cholesterol efflux to various forms of HDL, (2) esterification of HDL-cholesterol by lecithin:cholesterol acyltransferase (LCAT) and (3) hepatic uptake of mature HDL.

Cholesterol efflux: There are at least three mechanisms for efflux. One is mediated by microsolubilization of membrane lipids by apo A-I via interaction ABCA1, which triggers the unidirectional release of cholesterol and PL that forms nascent HDL (Gillotte et al., 1999; Vedhachalam et al., 2007; Okuhira et al., 2004). ABCG1 mediates efflux to HDL but not to LF apoA-I (Wang et al., 2004; Terasaka et al., 2007; Nakamura et al., 2004). ABCA1 and ABCG1 are both highly expressed in macrophages (Wang et al., 2004; Terasaka et al., 2007; Nakamura et al., 2004) and could mediate efflux from macrophage-foam cells to early forms of HDL. Cholesterol also spontaneously desorbs from cells into the surrounding aqueous phase where it associates with HDL. This process is driven by a cholesterol concentration gradient from high (donor) to low (acceptor); high relative levels of acceptor-sphingomyelin, which is highly cholesterophilic, increase efflux (Phillips et al., 1987; Phillips et al., 1998; Lund-Katz et al., 1988). SR-B1, which mediates selective hepatic removal of HDL-CE, -TG, and -PL (Acton et al., 1996), also mediates cholesterol efflux; efflux is enhanced by replacing acceptor-PC with the more cholesterophilic PL, sphingomyelin (Jian et al., 1997; Pownall, 2006; Yancey et al., 2000). The importance of SR-BI in macrophages is unresolved. Although phospholipids are the essential cholesterophilic component of all lipoproteins, including HDL (Jian et al., 1997; Pownall, 2006; Yancey et al., 2000), it is the magnitude of the free cholesterol to phospholipid ratio that is the quantitative determinant of net efflux; below 15 mol % cholesterol, rHDL effects a dose-dependent cellular efflux that triggers respective increases and decreases in HMG-CoA reductase and ACAT activities; above 15 mol % the opposite is observed (Picardo et al., 1986).

A therapeutic dilemma: There is no doubt that low plasma HDL-C is a CVD risk factor for which current therapies are inadequate. In the context of the current model in which HDL is the major RCT vehicle, therapeutic options are less clear, presenting a therapeutic dilemma—raise HDL or increase RCT irrespective of the effects on HDL-C.

Raise HDL: Several landmark studies have shown that high plasma HDL-C is cardioprotective. E.g. HDL-C and CVD are negatively correlated (Fricke t al., 1987; Gordon et al., 1977; Jacobs et al., 1990); raising HDL-C with a fibrate or niacin reduces events and/or lesion formation (Manninen et al., 1988; Robins, 2001; Brown et al., 2001; Zhao et al., 2004); transgenic mice over expressing apo A-I have increased HDL-C and less atherosclerosis than control animals (Rubin et al., 1991); increased plasma HDL-C mediates 50-90% of the cardioprotective effects of alcohol (Criqui et al., 1987; Thun et al., 1997); Although exercise is often prescribed for reduction of CVD risk, its effects are often confounded by concurrent weight loss and dietary changes. Nevertheless, exercise is likely cardioprotective with its effects being mediated by profound increases in HDL-C particularly in HDL₂(Patsch et al., 1983). Finally, some HDL deficiencies and attendant CVD are due to defects in a cellular cholesterol translocator, ABCA1 (Marcil, 1999). This transporter and ABCG1 “push” cholesterol into the extracellular space in response to LF and lipidated apo A-I respectively (Wang et al., 2004; Gillotte et al., 1999; Vedhachalam et al., 2007; Okuhira et al., 2004; Francis et al., 1995).

Increase RCT: Some HDL deficiencies and attendant CVD are due to defects in a cellular cholesterol translocator, ABCA1 (Farncis et al., 1995). This observation is in both the “raise HDL-C” and “increase RCT” camp. But there is additional evidence in support of treating impaired RCT. A common CETP gene mutation that lowers plasma CETP is associated with high HDL-C and possibly with increased CHD in HTG men (Zhong et al., 1996; Bruce et al., 1998). In murine models of atherosclerosis, hepatic over expression of SR-BI, the HDL receptor that “pulls” cholesterol out of extrahepatic spaces, decreases plasma HDL-C (Kozarsky et al., 1997; Wang et al., 1998; Ueda et al., 1999), increases HDL-CE clearance (Wang et al., 1998; Ueda et al., 1999; Ji et al., 1999), biliary cholesterol, and its transport into bile (Kozarsky et al., 1997; Ueda et al., 1999; Sehayek et al., 1998), but reduces atherosclerosis (Arai et al., 1999; Ueda et al., 2000; Kozarsky et al., 2000). Conversely, ablated or attenuated hepatic SR-BI expression elevates plasma HDL-C and reduces selective HDL-CE clearance (Ueda et al., 1999) but is atherogenic (Ueda et al., 1999; Covey et al., 2003; Huszar et al., 2000).

Current HDL Therapies: Even two frequently prescribed HDL therapies—fibrates and niacin—elicit other potentially cardioprotective effects that may be mechanistically more closely linked to cardioprotection. Niacin inhibits adipose tissue-lipolysis thereby reducing the amount of fatty acid available for hepatic extraction and triglyceride synthesis. Fibrates, PPARα agonists, increase hepatic fatty acid oxidation, again reducing triglyceride synthesis. While increasing HDL-C, both fibrates and niacin lower plasma VLDL. Although niacin and fibrates increase HDL-C and reduce CVD, more powerful therapies in combination with statins are needed to reverse atherosclerosis. Within the context of RCT and current understanding of the structure and properties of HDL, there are other obvious mechanistic RCT determinants that could be incorporated into a rational HDL therapy. The first step, efflux is enhanced by increasing HDL-PL (Pownal, 2006; Jian et al., Yancey et al., 2000). The underlying mechanism for this is that increased HDL-PL is also associated with a decrease in the FC/PL, an effect that shifts the equilibrium distribution of cholesterol from the plasma membrane to HDL (Picardo et al., 1986); this correlation is highly relevant to the neo HDL action, which in one embodiment better mediates the “push” of free cholesterol from macrophages to HDL via ABCA1, ABCG1, and spontaneous transfer. In another embodiment, HDL action will also utilize the HDL remodeling pathways of human plasma, especially LCAT which converts cholesterol to an ester that does not spontaneously transfer between lipid surfaces, while converting HDL from a disc to sphere with a CE core. In a specific embodiment, the therapy will enhance the CE “pull” step of RCT, hepatic CE disposal and prevent or reverse atherosclerosis.

HDL Stability and Apo A-I Lability—Keys to HDL Function and Rational Therapies? Physico-chemical probes reveal HDL stability and apo A-I lability. Chaotropic and detergent perturbation of HDL transfer LF apo A-I but not apo A-II to the aqueous phase while forming apo A-II-rich particles (Mehta et al., 2003; Pownall et al., 2005); this is not seen with LDL or VLDL and apo A-I appears to be a key determinant of how HDL is different, a difference that is also seen in the Interactions of HDL with its major remodeling activities—cholesteryl ester transfer protein, lecithin:cholesterol acyltransferase, and especially phospholipid transfer protein all of which liberate LF apo A-I (Rye et al., 1997; Liang et al., 1996; Rao et al., 1997; Lusa et al., 1996; Silver et al., 1990). Thus, special properties of apo A-I that determine its stability and metabolism (Curtiss et al., 2006; Pownall and Ehnholm, 2006) provide the context that makes the activity of a bacterial fusogen that targets HDL scientifically provocative and medically relevant.

### Example 26

### Exemplary Summary

The following is an exemplary summary of the previous examples.

SOF, a Bacterial Fusogen that Targets and Disrupts Human HDL: SOF is a virulence determinant expressed by approximately half of the clinical isolates of S. pyogenes, a human pathogen that causes a spectrum of diseases ranging from pharyngitis to overwhelming invasive infections with high rates of morbidity and mortality (Cunnignham, 2000). The target of opacification is HDL; other lipoproteins are not substantively affected. rSOF opacifies HDL without breaking covalent bonds and is neither a protease nor a lipase (Courtney et al., 2006). The products of SOF activity are buoyant lipid droplets that are devoid of apos and a denser fraction that is rich in apos A-I and A-II. SOF appears to interact with HDL-apos A-I and A-II, thereby triggering the extrusion of HDL lipids, which coalesce into lipid droplets that are the source of opacification (Courtney et al., 2006).

Identification of the mechanism by which any substance or process selectively delipidates HDL is of interest because the accruing insights may help identify additional therapeutic modalities. Using a recombinant (r) SOF, opacification and its mechanism was studied. rSOF catalyzes the partial disproportionation of HDL into a CERM and a new HDL-like particle, neo HDL, with the concomitant release of lipid-free (LF)-apo A-I (Gillard et al., 2007).

Opacification is unique; rSOF transfers apo E and nearly all neutral lipids of ˜100,000 HDL particles into a single large CERM whose size increases with HDL-CE content (r ˜100-250 nm) leaving a neo HDL that is rich in PL (41%) and protein (48%), especially apo A-II, and a lower FC/PL ratio that HDL (Gillard et al., 2007).

rSOF is potent; within 30 min at 37 EC, 10 nM rSOF opacifies 4 ΦM HDL (Gillard et al., 2007).

rSOF is catalytic: total opacification occurs with a >500 ratio of HDL particles to rSOF molecules.

CERM formation and apo A-I release have similar kinetics suggesting parallel or rapid sequential steps (Gillard et al., 2007).

According to the kinetic studies, rSOF is a heterodivalent fusogen that uses a high affinity docking site to displace apo A-I and bind to exposed CE on HDL; the rSOF-HDL complex recruits additional HDL with its binding-delipidation site and through multiple fusion steps forms a CERM.

The SOF Reaction—a Novel and Clinically Useful Modality for Improving RCT: All of the products of HDL opacification-neo HDL, CERM, and LF apo A-I—have the potential to increase RCT.

The preliminary data showing that neo HDL, is a better acceptor of cholesterol efflux from THP-1 macrophages than HDL confirms that neo HDL with a free cholesterol content that is half that of HDL (6 vs. 12 mol %) and far below the 15 mol % “switch” would be a better acceptor of cellular cholesterol than HDL (Gillard et al., 2007; Examples 1-10). This was observed despite the fact that neo HDL is apo A-II-rich. Tests on macrophages from control mice and mice in which ABCA1 (ABCG1) have been ablated will be done. The excess of efflux from macrophages of control vs. KO mice corresponds that which is mediated by the ablated transporter. In one embodiment, ABCG1 is the most important efflux pathway to neo HDL because of its high expression in macrophages and its specificity to lipidated species (Wang et al., 2004; Terasaka et al., 2007; Nakamura et al., 2004).

In one embodiment, with apo E and a high CE content, CERM transfers large amounts of CE to the liver for disposal via the LDL receptor. Again as shown in the data, more CE is taken up by cultured hepatocytes via CERM and than by the HDL from which it was formed. Moreover, other data shows that apo A-II-rich HDL, which is similar to neo HDL is a better donor of CE to cells expressing SR-BI than the HDL from which it was derived. This work will be further tested in hepatocytes from LDL-R KO and SR-BI KO mice.

rSOF also forms LF apo A-I, the ligand for cholesterol efflux via ABCA1. Impaired cholesterol transport via ABCA1 produces a low HDL-C, atherogenic state, and in its severest manifestation, Tangier disease, which is characterized by the total absence of normal HDL particles (Francis et al., 2004; Brooks-Wilson et al., 1999). It is not known whether low plasma LF apo A-I is atherogenic or whether increasing its plasma concentration is atheroprotective. The evidence is sparse and equivocal. Low apo A-I correlates with CVD risk and it is possible, but remains to be shown that the plasma concentration of LF apo A-I is correspondingly reduced. Although the plasma and perhaps macrophage (Curtiss et al., 2006) activities of LCAT, -CETP, and especially -PLTP produce LF apo A-I (Rye et al., 1997; Liang et al., 1996; Rao et al., 1997; Lusa et al., 1996; Siler et al., 1990; Curtiss et al., 2006; Pownall and Ehnholm, 2006), according to SEC studies, there is very little in human plasma. In one embodiment this is due to rapid in vivo lipidation via ABCA1. In a further embodiment, the effects of low HDL and apo A-I produce an atherogenic state through low LF apo A-I and the rSOF pathway provides a means for its enhancement.

### Example 27

### Cell and Mouse Models

The following are exemplary studies for the use of SOF and SOF-generated anti-astherosclerosis therapeutic particles in cell and mouse models.

Quantification of RCT In Vivo: Robust assays for the major RCT steps—cellular cholesterol efflux, cholesterol esterification, remodeling by lipid transfer proteins, and selective uptake—are well known to one of skill in the art. Until recently, there were no reliable assays for measuring the entire RCT pathway in vivo, a serious deficiency if one is to test new HDL therapies. Rader and co workers developed a method of RCT quantification (Zhang et al 2003, 2005). J774 macrophages are loaded with [³H]cholesterol by incubation with [³H]cholesterol-labeled acetylated LDL and injected intraperitoneally into mice. At various times, plasma, liver, and feces are collected and analyzed for [³H]cholesterol. Using this model, they showed enhanced RCT in two mouse models of cardioprotection. Apo A-I over expression led to higher [³H]cholesterol in plasma, liver, and feces (Zhang et al 2003). Hepatic SR-BI over expression (deficiency) reduced (increased) [³H]cholesterol in the plasma but markedly increased (lower) [³H] tracer in feces over a 48-hour interval. Interestingly, the SR-BI mouse models indicate enhanced (reduced) RCT in the presence of reduced (higher) plasma [³H]cholesterol (Zhang et al. 2005). In one embodiment of the invention, neo HDL and/or CERM will inhibit or reverse atherogenesis by reducing the cholesterol burden on arterial macrophages. Uptake and metabolism of HDL-CE with that of CERM-CE by hepatocytes from WT, LDL-receptor KO, and SR-BI KO mice will be compared. Also compared will be the transfer of peritoneal macrophage-cholesterol to plasma, liver and feces in mice treated with neo HDL with those treated with native HDL, rHDL, and control saline.

Regression/Prevention of Atherosclerosis: There are several mouse models of atherosclerosis that will be used to test atheroprotection. These include the apo bec/LDL-receptor double KO (Dutta et al., 2003; Singh et al., 2004), the apo A-I KO, apo E KO, cystathionine beta-synthase and apolipoprotein E, the apo bec/LDL-receptor double KO, and the apo A-I/SR-BI double KO. Although the apo E KO mouse is the best characterized and frequently used to test the effects of various interventions on atherogenesis, it may not be ideal for studying the anti-atherogenic effects of improved efflux. Several studies have shown that macrophage apo E is essential for the optimal FC efflux to HDL and HDL-like species, which would include neo HDL (Lin et al., 19099; Lin et al., 2001; Huang et al., 2001). Thus, LDLR−/− mice and LDb mice will be used, which are deficient in both LDL receptors and the apo B editing enzyme, apobec. The lipoprotein profiles of LDb mice simulate those of human familial hypercholesterolemia; the mice develop atherosclerotic lesions on a chow diet by 8-month of age and a Western-type high-fat diet induces higher total plasma and LDL cholesterol, and more severe atherosclerotic lesions that are positive for lipid (Oil Red O), calcium (Alizarin Z) and macrophages (Mac-1α chain, CD11b) than those on chow. The mice are viable for more than a year and according to western blot analysis, there is no difference in the hepatic expression of SR-BI when compared to background C57B1 mice (Dutta et al., 2003; Singh et al., 2004).

LDLR−/− mice and LDb mice will be treated with SOF by injection, oral dosage, or ex vivo treatment. Measurement of blood cholesterol levels after treatment with SOF will show an immediate decrease in total plasma cholesterol and an increased amount of neo HDL and the subsequent appearance of mature forms of HDL, which contains cholesterol extracted from peripheral tissue over a period of 12 to 36 hours.

### Example 28

### An Example of Treatment

In one instance of the invention, an individual will seek medical treatment of atherosclerosis, or a condition caused from advanced atherosclerosis such as heart attack, stroke, or peripheral arterial disease. After seeking medical attention, this individual will receive an injection of SOF in a suitable pharmaceutical carrier at a dosage of 3 mg/75 kg, for example, a pill containing SOF in a pharmaceutical acceptable carrier at a dosage of 3 mg/75 kg, for example, and/or receive ex vivo treatment in which blood is drawn and run over SOF attached to a solid support and then re-injected into the patient. Measurement of blood cholesterol levels after treatment with SOF will show an immediate decrease in total plasma cholesterol and an increased amount of neo HDL and the subsequent appearance of mature forms of HDL, which contains cholesterol extracted from peripheral tissue over a period of 12 to 36 hours.

All patents and publications cited herein are hereby incorporated by reference in their entirety herein. Full citations for the references cited herein are provided in the following list.

### PUBLICATIONS

U.S. Pat. No. 4,554,101
U.S. Pat. No. 5,399,363
U.S. Pat. No. 5,446,128
U.S. Pat. No. 5,466,468
U.S. Pat. No. 5,543,158
U.S. Pat. No. 5,580,579
U.S. Pat. No. 5,629,001
U.S. Pat. No. 5,641,515
U.S. Pat. No. 5,440,013
U.S. Pat. No. 5,475,085
U.S. Pat. No. 5,618,914
U.S. Pat. No. 5,670,155
U.S. Pat. No. 5,672,681
U.S. Pat. No. 5,674,976.
U.S. Pat. No. 5,710,245
U.S. Pat. No. 5,725,871
U.S. Pat. No. 5,756,353
U.S. Pat. No. 5,780,045
U.S. Pat. No. 5,792,451
U.S. Pat. No. 5,804,212
U.S. Pat. No. 5,840,833
U.S. Pat. No. 5,859,184
U.S. Pat. No. 5,929,237
U.S. Pat. No. 6,613,308
Acton, S., Rigotti, A., Landschulz, K. T., Xu, S., Hobbs, H. H., Krieger, M. (1996) Identification of scavenger receptor SR-BI as a high density lipoprotein receptor. Science 271, 518-520.
Arai, T., Wang, N., Bezouevski, M., Welch, C., Tall, A. R. (1999) Decreased atherosclerosis in heterozygous low density lipoprotein receptor-deficient mice expressing the scavenger receptor BI transgene. J. Biol. Chem. 274, 2366-2371.
B. F. Asztalos, E. J. Schaefer, K. V. Horvath, S. Yamashita, M. Miller, G. Franceschini, and L. Calabresi, 2007 Role of LCAT in HDL remodeling: investigation of LCAT deficiency states. J Lipid Res. 48, 592-9.
Braun, A., Trigatti, B. L., Post, M. J., Sato, K., Simons, M., Edelberg, J. M., Rosenberg, R. D., Schrenzel, M., Krieger, M. (2002) Loss of SR-BI expression leads to the early onset of occlusive atherosclerotic coronary artery disease, spontaneous myocardial infarctions, severe cardiac dysfunction, and premature death in apolipoprotein E-deficient mice. Circ. Res. 90, 270-276.
H. B. Brewer Jr, R. Ronan, M. Meng, C. Bishop, 1986 Isolation and characterization of apolipoproteins A-I, A-II, and A-IV. Methods Enzymol. 128, 223-46.
Brooks-Wilson A, Marcil M, Clee S M, et al Mutations in ABC1 in Tangier disease and familial high-density lipoprotein deficiency. Nat. Genet. 1999 22:336-45.
Brinton, E. A., Eisenberg, S., and Breslow, J. L. (1991) Increased apo A-I and apo A-II fractional catabolic rate in patients with low high density lipoprotein-cholesterol levels with or without hypertriglyceridemia, J. Clin. Invest. 87, 536-544.
Brown B G, Zhao X Q, Chait A, et al. Simvastatin and niacin, antioxidant vitamins, or the combination for the prevention of coronary disease. N Engl J. Med. 2001 Nov. 29; 345(22):1583-1592.
Bruce C, Sharp D S, Tall A R. Relationship of HDL and coronary heart disease to a common amino acid polymorphism in the cholesteryl ester transfer protein in men with and without hypertriglyceridemia. J Lipid Res. 1998 39:1071-8.
G. R. Castro, C. J. Fielding. 1988 Early incorporation of cell-derived cholesterol into pre-beta-migrating high-density lipoprotein. Biochemistry. 27, 25-9.
Courtney, H. S., Hasty, D. L., Li, Y., Chiang, H. C., Thacker, J. L., Dale, J. B. (1999) Serum opacity factor is a major fibronectin-binding protein and a virulence determinant of M type 2 Streptococcus pyogenes. Mol. Microbiol. 32, 89-98.
Courtney, H. S., Zhang, Y. M., Frank, M. W., Rock, C. O. (2006) Serum opacity factor, a streptococcal virulence factor that binds to apolipoproteins A-I and A-II and disrupts high density lipoprotein structure. J. Biol. Chem. 281, 5515-5521.
Covey, S. D., Krieger, M., Wang, W., Penman, M., Trigatti, B. L. (2003) Scavenger receptor class B type I-mediated protection against atherosclerosis in LDL receptor-negative mice involves its expression in bone marrow-derived cells. Arterioscler. Thromb. Vasc. Biol. 23, 1589-1594.
Criqui et al. 1987 Lipoproteins as mediators for the effects of alcohol consumption and cigarette smoking on cardiovascular mortality: results form the LRC Follow-up Study. Am J Epidemiol; 126:629-37.
Cunningham, M. W. (2000). Pathogenesis of group A streptococcal infections. Clin. Microbiol. Rev. 13, 470-511.
Curtiss L. K., Valenta D. T., Hime N. J., & Rye K. A. What is so special about apolipoprotein AI in reverse cholesterol transport? Arterioscler. Thromb. Vasc. Biol. 2006 January; 26(1):12-9.
Cuchel, M. and Rader, D. J., 2006 Macrophage reverse cholesterol transport: key to the regression of atherosclerosis? Circulation. 113, 2548-55.
W. S. Davidson, W. V. Rodrigueza, S. Lund-Katz, W. J. Johnson, G. H. Rothblat, and M. C. Phillips, 1995 Effects of acceptor particle size on the efflux of cellular free cholesterol. J. Biol. Chem. 270, 17106-113.
P. T. Duong, H. L. Collins, M. Nickel, S. Lund-Katz, G. H. Rothblat, M. C. Phillips, 2006 Characterization of nascent HDL particles and microparticles formed by ABCA1-mediated efflux of cellular lipids to apoA-I. J Lipid Res. 47, 832-43.
Dutta R, Singh U, Li T B, Formage M, Teng B B. Hepatic gene expression profiling reveals perturbed calcium signaling in a mouse model lacking both LDL receptor and Apobecl genes. Atherosclerosis. 2003 169:51-62.
H. Edelhoch, 1967 Spectroscopic determination of tryptophan and tyrosine in proteins. Biochemistry. 6, 1948-54.
N. Fournier, M. de la Llera Moya, B. F. Burkey, J. B. Swaney, J. Paterniti Jr, N. Moatti, V. Atger, G. H. Rothblat. 1996 Role of HDL phospholipid in efflux of cell cholesterol to whole serum: studies with human apoA-I transgenic rats. J Lipid Res. 37, 1704-11.
N. Fournier, J. L. Paul, V. Atger, A. Cogny, T. Soni, M. de la Llera-Moya, G. H. Rothblat, N. Moatti, 1997 HDL phospholipid content and composition as a major factor determining cholesterol efflux capacity from Fu5AH cells to human serum. Arterioscler. Thromb. Vasc. Biol. 17, 2685-91.
Francis G A, Knopp R H, Oram J F. Defective removal of cellular cholesterol and phospholipids by apolipoprotein A-I in Tangier Disease. J Clin Invest. 1995 96:78-87.
Frick M. H., Elo O., Haapa K., Heinonen O. P., Heinsalmi P., Helo P., Huttunen J. K., Kaitaniemi P., Koskinen P., Manninen V., et al. (1987) Helsinki Heart Study: primary-prevention trial with gemfibrozil in middle-aged men with dyslipidemia. Safety of treatment, changes in risk factors, and incidence of coronary heart disease. N. Engl. J. Med. 317, 1237-45.
J. W. Gaubatz, B. K. Gillard, J. B. Massey, R. C. Hoogeveen, M. Huang, E. E. Lloyd, J. L. Raya, C. Y. Yang, and H. J. Pownall, 2007 Dynamics of dense electronegative low density lipoproteins and their preferential association with lipoprotein phospholipase A(2). J Lipid Res. 48, 348-57.
Gaus K, Gooding J J, Dean R T, Kritharides L, Jessup W. A kinetic model to evaluate cholesterol efflux from THP-1 macrophages to apolipoprotein A-1. Biochemistry. 2001 40:9363-73.
Gillard B K, Courtney H S, Massey J B, & Pownall H J. Serum Opacity Factor Unmasks Human Plasma High Density Lipoprotein Instability via Selective Delipidation and Apolipoprotein A-I Desorption. 2007 Biochemistry 2007 46:12968-12978.
Glass, C., Pittman, R. C., Weinstein, D. B., Steinberg, D. (1983) Dissociation of tissue uptake of cholesterol ester from that of apoprotein A-I of rat plasma high density lipoprotein: selective delivery of cholesterol ester to liver, adrenal, and gonad. Proc. Natl. Acad. Sci. U.S.A. 80, 5435-5439.
Gillotte K L, Zaiou M, Lund-Katz S, Anantharamaiah G M, Holvoet P, Dhoest A, Palgunachari M N, Segrest J P, Weisgraber K H, Rothblat G H, Phillips M C. Apolipoprotein-mediated plasma membrane microsolubilization. Role of lipid affinity and membrane penetration in the efflux of cellular cholesterol and phospholipid. J Biol. Chem. 1999 274:2021-8.
Gordon, T., Hjortland, M., Kannel, W., & Dawber, T. (1977) High density lipoprotein as a protective factor against coronary heart disease: the Framingham Study. Am. J. Med. 66, 707-714.
Gotto, A. M., Jr and Pownall, H. J. (2003). Manual of Lipid Disorders (3rd Ed). Lippincott Williams and Wilkins. Baltimore. 462 p.
Gotto, A. M. Jr., Pownall, H. J., and R. J. Havel. (1986) Plasma lipoproteins. In Methods in Enzymology (J. Segrest and J. Albers, eds.) 128, 3-41.
Hamilton, J. A., Miller, K. W., Small, D. M. (1983) Solubilization of triolein and cholesteryl oleate in egg phosphatidylcholine vesicles. J. Biol. Chem. 258, 12821-6.
Havel, R. J., Goldstein, J. L., and Brown, M. S. (1980) Lipoproteins in Lipid Transport. In The Metabolic Control of Disease (P. K. Bondy and L. E. Rosenberg, Ed): 398-494.
Huang Z H, Lin C Y, Oram J F, Mazzone T. Sterol efflux mediated by endogenous macrophage ApoE expression is independent of ABCA1. Arterioscler Thromb Vasc Biol. 2001 21:2019-25.
Huszar D, Varban M L, Rinninger F, Feeley R, Arai T, Fairchild-Huntress V, Donovan M J, Tall A R. Increased LDL cholesterol and atherosclerosis in LDL receptor-deficient mice with attenuated expression of scavenger receptor B1. Arterioscler Thromb Vasc Biol. 2000 20:1068-1073.
Jacobs, D. R., Mebane, I. L., Bangdiwal, S. I., Criqui, M. H., and Tyroler, H. A. (1990) High density lipoprotein cholesterol as a predictor of cardiovascular disease mortality in men and women: The Follow-up Study of the Lipid Research Clinics Prevalence Study. Am. J. Epidemiol. 131, 32-47.
Ji Y, Wang N, Ramakrishnan R, Sehayek E, Huszar D, Breslow J L, Tall A R. Hepatic scavenger receptor BI promotes rapid clearance of high density lipoprotein free cholesterol and its transport into bile. J Biol. Chem. 1999 274:33398-33402.
Jian B, de la Llera-Moya M, Royer L, Rothblat G, Francone O, Swaney J B. Modification of the cholesterol efflux properties of human serum by enrichment with phospholipid. J Lipid Res. 1997 38:734-44.
A. Jonas, N. L. Zorich, K. E. Kézdy, W. E. Trick, 1987 Reaction of discoidal complexes of apolipoprotein A-I and various phosphatidylcholines with lecithin cholesterol acyltransferase. Interfacial effects. J Biol. Chem. 262, 3969-74.
Kozarsky, K. F., Donahee, M. H., Rigotti, A., Iqbal, S. N., Edelman, E. R., Krieger, M. (1997) Overexpression of the HDL receptor SR-BI alters plasma HDL and bile cholesterol levels. Nature 387, 414-417.
Kozarsky K F, Donahee M H, Glick J M, Krieger M, Rader D J. Gene transfer and hepatic overexpression of the HDL receptor SR-BI reduces atherosclerosis in the cholesterol-fed LDL receptor-deficient mouse. Arterioscler Thromb Vasc Biol. 2000 20:721-727.
Lawrence. (2007) Essentials of surgical specialties. Lippincott Williams & Wilkins, Baltimore Md.
Liang, H. Q., Rye, K. A., Barter, P. J. (1996) Remodelling of reconstituted high density lipoproteins by lecithin: cholesterol acyltransferase. J. Lipid Res. 37, 1962-1970.
Lin C Y, Duan H, Mazzone T. Apolipoprotein E-dependent cholesterol efflux from macrophages: kinetic study and divergent mechanisms for endogenous versus exogenous apolipoprotein E. J Lipid Res. 1999 40:1618-27.
Lin C Y, Huang Z H, Mazzone T. Interaction with proteoglycans enhances the sterol efflux produced by endogenous expression of macrophage apoE. J Lipid Res. 2001 42:1125-33.
Ludtke S. J., Baldwin P. R., & Chiu W. EMAN: semiautomated software for high-resolution single-particle reconstructions. J. Struct. Biol. 1999 128:82-97.
Lund-Katz S, Laboda H M, McLean L R, Phillips M C. Influence of molecular packing and phospholipid type on rates of cholesterol exchange. Biochemistry. 1988 27:3416-23.
Lusa, S., Jauhiainen, M., Mets, J., Somerharju, P., Ehnholm, C. (1996) The mechanism of human plasma phospholipid transfer protein-induced enlargement of high-density lipoprotein particles: evidence for particle fusion. Biochem. J. 313 (Pt 1), 275-82.
Manninen V., Elo M. O., Frick M., et al. (1988) Lipid alterations and decline in the incidence of coronary heart disease in the Helsinki Heart Study. JAMA. 260, 641-51.
W. W. Mantulin, J. B. Massey, A. M. Gotto, Jr., and H. J. Pownall, 1981 Reassembled model lipoproteins: lipid dynamics in recombinants of human apolipoprotein A-II and dimyristoylphosphatidylcholine. J. Biol. Chem. 256, 10815-19.
J. B. Massey, 2001 Interaction of ceramides with phosphatidylcholine, sphingomyelin and sphingomyelin/cholesterol bilayers. Biochim Biophys Acta. 1510, 167-84.
Massey, J. B., Hickson, D., She, H. S., Sparrow J. T., Via D. P., Gotto A. M. Jr, Pownall H. J. (1984) Measurement and prediction of the rates of spontaneous transfer of phospholipids between plasma lipoproteins. Biochim. Biophys. Acta 794, 274-280.
J. B. Massey, and H. J. Pownall, 1998 Surface properties of native human plasma lipoproteins and lipoprotein models. Biophys J. 74:869-78.
J. B. Massey, H. S. She, A. M. Gotto Jr, and H. J. Pownall, 1985a Lateral distribution of phospholipid and cholesterol in apolipoprotein A-I recombinants. Biochemistry. 24:7110-6.
J. B. Massey, H. S. She, and H. J. Pownall. 1985b Interfacial properties of model membranes and plasma lipoproteins containing ether lipids. Biochemistry. 24, 6973-8.
J. B. Massey, H. S. She, A. M. Gotto, Jr., and H. J. Pownall, 1985c Lateral distribution of phospholipid and cholesterol in apolipoprotein A-I recombinants. Biochemistry. 24:7110-6.
J. B. Massey, and H. J. Pownall, 2005 The polar nature of 7-ketocholesterol determines its location within membrane domains and the kinetics of membrane microsolubilization by apolipoprotein A-I. Biochemistry. 44, 10423-33.
J. B. Massey, and H. J. Pownall, 2006 Structures of biologically active oxysterols determine their differential effects on phospholipid membranes. Biochemistry. 45, 10747-58.
Marcil 1999 Mutations in the ABC1 gene in familial HDL deficiency with defective cholesterol efflux. Lancet. 354:1341.
Mehta, R., Gantz, D. L., Gursky, O. (2003) Human plasma high-density lipoproteins are stabilized by kinetic factors. J. Mol. Biol. 328, 183-192.
Miller, N. E., Thelle, D. S., Førde, O. H., & Mjøs, O. D. (1977) The Tromsø heart study: high-density lipoprotein and coronary heart-disease: a prospective case-control study. Lancet 1, 964-968
Nakamura K, Kennedy M A, Baldan A, Bojanic D D, Lyons K, Edwards P A. Expression and regulation of multiple murine ATP-binding cassette transporter G1 mRNAs/isoforms that stimulate cellular cholesterol efflux to high density lipoprotein. J Biol. Chem. 2004 279:45980-9.
Okuhira K, Tsujita M, Yamauchi Y, Abe-Dohmae S, Kato K, Handa T, Yokoyama S. Potential involvement of dissociated apoA-I in the ABCA1-dependent cellular lipid release by HDL. J Lipid Res. 2004 45:645-52.
Oram, J. F., Lawn, R. M., Garvin, M. R., Wade, D. P. (2000) ABCA1 is the cAMP-inducible apolipoprotein receptor that mediates cholesterol secretion from macrophages. J. Biol. Chem. 275, 34508-34511.
J. F. Oram, A. M. Vaughan, 2006 ATP-Binding cassette cholesterol transporters and cardiovascular disease. Circ Res. 99, 1031-43.
T. Parasassi, M. Di Stefano, M. Loiero, G. Ravagnan, E. Gratton, 1994 Cholesterol modifies water concentration and dynamics in phospholipid bilayers: a fluorescence study using Laurdan probe. Biophys J. 66, 763-8.
T. Parasassi, G. De Stasio, A. d'Ubaldo, and E. Gratton. 1990. Phase fluctuation in phospholipid membranes revealed by Laurdan fluorescence. Biophys. J. 57, 1179-1186.
T. Parasassi, G. De Stasio, G. Ravagnan, R. M. Rusch, and E. Gratton. 1991. Quantitation of lipid phases in phospholipid vesicles by the generalized polarization of Laurdan fluorescence. Biophys. J. 60, 179-180.
Patsch J R, Karlin J B, Scott L W, Smith L C, Gotto A M Jr. Inverse relationship between blood levels of HDL₂ and magnitude of postprandial lipemia. Proc Natl Acad Sci USA. 1983 80:1449-53.
Phillips M C, Johnson W J, Rothblat G H. Mechanisms and consequences of cellular cholesterol exchange and transfer. Biochim Biophys Acta. 1987 906:223-76.
Phillips M C, Gillotte K L, Haynes M P, Johnson W J, Lund-Katz S, Rothblat G H. Mechanisms of high density lipoprotein-mediated efflux of cholesterol from cell plasma membranes. Atherosclerosis. 1998 April; 137 Suppl:S13-7.
M. Picardo, J. B. Massey, D. E. Kuhn, A. M. Gotto Jr, S. H. Gianturco, and H. J. Pownall, 1986 Partially reassembled high density lipoproteins. Effects on cholesterol flux, synthesis, and esterification in normal human skin fibroblasts. Arteriosclerosis. 6, 434-41.
F. G. Prendergast, R. P. Haugland, P. J. Callahan, 1981 1-[4-(Trimethylamino)phenyl]-6-phenylhexa-1,3,5-triene: synthesis, fluorescence properties, and use as a fluorescence probe of lipid bilayers. Biochemistry. 20, 7333-8.
Pownall, H. J. Remodeling of human plasma lipoproteins by detergent perturbation. (2005) Biochemistry 44, 9714-9722.
Pownall, H. J. (2006) Detergent-mediated phospholipidation of plasma lipoproteins increases HDL cholesterophilicity and cholesterol efflux via SR-BI. Biochemistry 45, 11514-22.
Pownall H J, and Ehnholm C. The Unique Role of Apo A-I in HDL Remodeling and Metabolism. Current Opinion in Lipidology 2006, 17:209-213.
Pownall, H. J., Hosken, B. D., Gillard, B. K., Higgins, C. L., Lin, H. Y., and Massey, J. B. (2007) Speciation of Human Plasma High Density Lipoprotein: HDL Stability and Apolipoprotein A-I Partitioning. Biochemistry 46, 7449-7459.
H. J. Pownall, Q. Pao, J. B. Massey, 1985 Acyl chain and headgroup specificity of human plasma lecithin:cholesterol acyltransferase. Separation of matrix and molecular specificities. J. Biol. Chem. 260, 2146-52.
Rao, R., Albers, J. J., Wolfbauer, G., Pownall, H. J. (1997) Molecular and macromolecular specificity of human plasma phospholipid transfer protein. Biochemistry 36, 3645-3653.
Reijngoud, D.-J., and Phillips, M. C. (1984) Mechanism of dissociation of human apolipoprotein A-I, A-II, and C from complexes of dimyristoylphosphatidylcholine as studied by thermal denaturation. Biochemistry 23, 726-734.
Remington's Pharmaceutical Sciences, 18th Ed. Mack Printing Company, 1990.
A. Rhoads, G. G., Gulbrandssen, C. L., and Kagan, A. (1976) Serum lipoproteins and coronary heart disease in a population study of Hawaii Japanese men. N. Eng. J. Med. 294, 293-298
Robins 2001 HDL Intervention Trial. Relation of gemfibrozil treatment and lipid levels with major coronary events: VA-HIT: a randomized controlled trial. JAMA 285:1585-91.
Rubin E M et al. 1991 Inhibition of early atherogenesis in transgenic mice by human apo AI. Nature. 353:265-7.
Rye, K. A., Hime, N. J., Barter, P. J. (1997) Evidence that cholesteryl ester transfer protein-mediated reductions in reconstituted high density lipoprotein size involve particle fusion. J. Biol. Chem. 272, 3953-60.
V. N. Schumaker, and D. L. Puppione, 1986 Sequential flotation ultracentrifugation. Methods Enzymol. 128, 155-70.
Sehayek E, Ono J G, Shefer S, Nguyen L B, Wang N, Batta A K, Salen G, Smith J D, Tall A R, Breslow J L. Biliary cholesterol excretion: a novel mechanism that regulates dietary cholesterol absorption. Proc Natl Acad Sci USA. 1998 95:10194-10199.
Silver, E. T., Scraba, D. G., Ryan, R. O. (1990) Lipid transfer particle-induced transformation of human high density lipoprotein into apolipoprotein A-1-deficient low density particles. J. Biol. Chem. 265, 22487-22492.
Singh U, Zhong S, Xiong M, Li T B, Sniderman A, Teng B B. Increased plasma non-esterified fatty acids and platelet-activating factor acetylhydrolase are associated with susceptibility to atherosclerosis in mice. Clin Sci (Lond). 2004 106:421-32.
G. Simard, D. Loiseau, A. Girault, B. Perret. 1989 Reactivity of HDL subfractions towards lecithin-cholesterol acyltransferase. Modulation by their content in free cholesterol. Biochim. Biophys. Acta. 1005, 245-52.
Sparks, D. L., Phillips, M. C., Lund-Katz, S. (1992) The charge and structural stability of apolipoprotein A-I in discoidal and spherical recombinant high density lipoprotein particles. J. Biol. Chem. 267, 25839-25847.
Swenson, T. L., Brocia, R. W, Tall, A. R. (1988) Plasma cholesteryl ester transfer protein has binding sites for neutral lipids and phospholipids. J. Biol. Chem. 263, 5150-5157.
Swift, L. L., Kakkad, B., Boone, C., Jovanovska, A., Jerome, W. G., Mohler, P. J., Ong, D. E. (2005) Microsomal triglyceride transfer protein expression in adipocytes: a new component in fat metabolism. FEBS Lett. 579, 3183-3189.
Tall, A. (1995) Plasma lipid transfer proteins. Annu. Rev. Biochem. 64, 235-257.
Tanford, C. (1980) The Hydrophobic Effect: Formation of Micelles and Biological Membranes, pp 57, Wiley-Interscience, New York.
Terasaka N, Wang N, Yvan-Charvet L, Tall A R. High-density lipoprotein protects macrophages from oxidized low-density lipoprotein-induced apoptosis by promoting efflux of 7-ketocholesterol via ABCG1. Proc Natl Acad Sci USA. 2007 104:15093-8
Thun et al 1997 Alcohol consumption and mortality among middle-aged and elderly U.S. adults, NEJM 337:1705-14.
Trigatti, B., Rayburn, H., Vinals, M., Braun, A., Miettinen, H., Penman, M., Hertz, M., Schrenzel, M., Amigo, L., Rigotti, A., Krieger, M. (1999) Influence of the high density lipoprotein receptor SR-BI on reproductive and cardiovascular pathophysiology. Proc. Natl. Acad. Sci. U.S.A. 96, 9322-9327.
Ueda, Y., Gong, E., Royer, L., Cooper, P. N., Francone, O. L., Rubin, E. M. (2000) Relationship between expression levels and atherogenesis in scavenger receptor class B, type I transgenics. J. Biol. Chem. 275, 20368-73.
Ueda Y Royer L, Gong E, Zhang J, Cooper P N, Francone O, Rubin E M. Lower plasma levels and accelerated clearance of high density lipoprotein (HDL) and non-HDL cholesterol in scavenger receptor class B type I transgenic mice. J Biol. Chem. 1999 274:7165-7171.
Varban, M. L., Rinninger, F., Wang, N., Fairchild-Huntress, V., Dunmore, J. H., Fang, Q., Gosselin, M. L., Dixon, K. L., Deeds, J. D., Acton, S. L., Tall, A. R., Huszar, D. (1998) Targeted mutation reveals a central role for SR-BI in hepatic selective uptake of high density lipoprotein cholesterol. Proc. Natl. Acad. Sci. U.S.A. 95, 4619-4624.
Vedhachalam C, Duong P T, Nickel M, Nguyen D, Dhanasekaran P, Saito H, Rothblat G H, Lund-Katz S, Phillips M C. Mechanism of ATP-binding cassette transporter A1-mediated cellular lipid efflux to apolipoprotein A-I and formation of high density lipoprotein particles. J Biol. Chem. 2007 282:25123-30.
Vitello, L. B., and Scanu, A. M. (1976) Studies on human serumhigh density lipoproteins. Self-association of apolipoprotein A-Iin aqueous solutions, J. Biol. Chem. 251, 1131-1136.
Wang N, Lan D, Chen W, Matsuura F, Tall A R. ATP-binding cassette transporters G1 and G4 mediate cellular cholesterol efflux to high-density lipoproteins. Proc Natl Acad Sci USA. 2004 101:9774-9.
Wang N, Arai T, Ji Y, Rinninger F, Tall A R. Liver-specific overexpression of scavenger receptor BI decreases levels of very low density lipoprotein ApoB, low density lipoprotein ApoB, and high density lipoprotein in transgenic mice. J Biol. Chem. 1998 273:32920-32926.
Wetterau, J. R., Lin, M. C., and Jamil, H. (1997) Microsomal triglyceride transfer protein. Biochim. Biophys. Acta 1345, 136-150.
Yancey P G, de la Llera-Moya M, Swarnakar S, Monzo P, Klein S M, Connelly M A, Johnson W J, Williams D L, Rothblat G H. High density lipoprotein phospholipid composition is a major determinant of the bi-directional flux and net movement of cellular free cholesterol mediated by scavenger receptor BI. J Biol. Chem. 2000 275:36596-604.
Zhang Y, Zanotti I, Reilly M P, Glick J M, Rothblat G H, Rader D J. Overexpression of apolipoprotein A-I promotes reverse transport of cholesterol from macrophages to feces in vivo. Circulation. 2003 108:661-3.
Zang Y, Da Silva J R, Reilly M, Billheimer J T, Rothblat G H, Rader D J. Hepatic expression of scavenger receptor class B type I (SR-BI) is a positive regulator of macrophage reverse cholesterol transport in vivo. J Clin Invest. 2005 115:2870-4.
Zhao X Q, Morse J S, Dowdy A A, Heise N, DeAngelis D, Frohlich J, Chait A, Albers J J, Brown B G. Safety and tolerability of simvastatin plus niacin in patients with coronary artery disease and low high-density lipoprotein cholesterol (The HDL Atherosclerosis Treatment Study). Am J. Cardiol. 2004 93:307-12.
Zhong S, Sharp D S, Grove J S, Bruce C, Yano K, Curb J D, Tall A R. Increased coronary heart disease in Japanese-American men with mutation in the cholesteryl ester transfer protein gene despite increased HDL levels. J Clin Invest. 1996 97:2917-23.

What is claimed is:

1. A method of altering reverse cholesterol transport in an individual that has atherosclerosis or is at risk for atherosclerosis, comprising the step of delivering a therapeutically effective amount of serum opacity factor to the individual.

2. The method of claim 1, wherein the serum opacity factor is recombinant serum opacity factor.

3. The method of claim 2, wherein the recombinant serum opacity factor is not full-length serum opacity factor.

4. The method of claim 3, wherein the serum opacity factor lacks at least one region or domain selected from the group consisting of a fibronectin binding site, a leader sequence, Fn-binding repeats, a LPASG anchor, or any combination thereof.

5. The method of claim 1, wherein the delivery is in vivo.

6. The method of claim 5, wherein the serum opacity factor is injected into the individual at least once.

7. The method of claim 1, wherein the delivery is ex vivo.

8. The method of claim 7, wherein the serum opacity factor is attached to a solid support and the plasma, blood, serum, or isolated HDL of the individual is passed over the support at least once.

9. The method of claim 1, wherein the individual has received, will receive, or is receiving treatment for atherosclerosis.

10. The method of claim 9, wherein the treatment comprises a cholesterol-lowering drug, an anti-platelet drug, an anticoagulant, angioplasty with or without a stent, or surgery.

11. A method of generating therapeutic lipoprotein particles for an individual with atherosclerosis, comprising the step of delivering an effective amount of serum opacity factor to the individual.

12. The method of claim 11, wherein the serum opacity factor is recombinant serum opacity factor.

13. The method of claim 12, wherein the recombinant serum opacity factor is not full-length serum opacity factor.

14. The method of claim 13, wherein the serum opacity factor lacks at least one region or domain selected from the group consisting of a fibronectin binding site, a leader sequence, Fn-binding repeats, a LPASG anchor, or any combination thereof.

15. The method of claim 11, wherein the delivery is in vivo.

16. The method of claim 15, wherein the serum opacity factor is injected into the individual at least once.

17. The method of claim 11, wherein the delivery is ex vivo.

18. The method of claim 17, wherein the serum opacity factor is attached to a solid support and the plasma, blood, serum or isolated HDL of the individual is passed over the support at least once.

19. The method of claim 11, wherein the individual has received, will receive, or is receiving treatment for atherosclerosis.

20. The method of claim 19, wherein the treatment comprises a cholesterol-lowering drug, an anti-platelet drug, an anti-coagulant, surgery, angioplasty with or without a stent, or a combination thereof.

21. A kit for the treatment of atherosclerosis, comprising serum opacity factor housed in a suitable container.

22. The kit of claim 21, wherein the serum opacity factor is recombinant serum opacity factor

23. The kit of claim 22, further comprising an additional atherosclerosis treatment.

24. The kit of claim 23, wherein the additional atherosclerosis treatment comprises a cholesterol-lowering drug, an anti-platelet drug, an anti-coagulant, or a combination thereof.

25. The kit of claim 21, further comprising an ex vivo support.
